(19) United States
(12) Patent Application Publication
(10) Pub. No.: US 2026/0007764 A1
(43) Pub. Date: Jan. 8, 2026

(54) USE OF A CONJUGATE OF ANTI-CD47 ANTIBODY AND TOLL-LIKE RECEPTOR AGONIST

(71) Applicant: The Johns Hopkins University, Baltimore, MD (US)

(72) Inventors: Jung Soo Suk, Clarksville, MD (US); Seungwoo Chung, Baltimore, MD (US); Yunxuan Xie, Baltimore, MD (US)

(21) Appl. No.: 18/879,976

(22) PCT Filed: Jun. 30, 2023
(86) PCT No.: PCT/US23/26764

§ 371 Date: Dec. 30, 2024

Related U.S. Application Data
(60) Provisional application No. 63/357,455, filed on Jun. 30, 2022.

(51) Int. Cl. A61K 47/68; A61K 45/06; A61P 35/00; A61P 37/04; C07K 16/28

(52) U.S. Cl. CPC A61K 47/6807; A61K 45/06; A61K 47/6849; A61P 35/00; A61P 37/04; C07K 16/2803

(57) ABSTRACT

Methods of modulating an immune response for the treatment of cancer includes compositions which comprise chimeric molecules with an anti-CD47 binding domain and an immunomodulatory agent.

The present application claims the benefit of U.S. provisional application No. 63/357,455 filed Jun. 30, 2022, which is incorporated herein in its entirety.

### BACKGROUND

Cluster of Differentiation 47 (CD47), also known as integrin associated protein (IAP), is a transmembrane receptor belonging to the immunoglobulin superfamily of proteins. CD47 is ubiquitously expressed on cells and serves as a marker for self-recognition, preventing phagocytosis by serving as a “don't eat me” signal. CD47 mediates its effects through interactions with several other proteins, including thrombospondin (TSP) and signal regulatory protein-alpha (SIRPα). The interaction between SIRPα on phagocytic cells and CD47 on target cells helps ensure that target cells do not become engulfed.

Certain cancers co-opt the CD47-based immune evasion mechanism of a cell by increasing expression of CD47 on the cell surface of the cancer cell, thus avoiding clearance by the immune system. However, therapies known in the art that target CD47-expressing cells in a subject target both cancerous and non-cancerous cells, which leads to toxicities in the subject, such as peripheral red blood cell and platelet depletion. Accordingly, there is a need for compositions and methods to selectively target CD47 in cancer cells without targeting non-cancerous cells.

### SUMMARY

Embodiments are directed to compositions for the treatment of cancer and infectious diseases. In particular, compositions comprise chimeric molecule having an anti-CD47 domain and an immunomodulatory agent.

In one aspect, a method of treating cancer comprises administering to a subject in need thereof, a composition comprising a therapeutically effective amount of chimeric molecule comprising a CD47 binding domain and an immunomodulatory agent wherein the immunomodulatory agent modulates innate and/or adaptive immune response(s), thereby treating cancer. In certain embodiments, the CD47 binding domain comprises antibodies, antibody fragments, single chain antibodies, humanized antibodies and antibody fragments; camelized antibodies and antibody fragments, human antibodies and antibody fragments, monospecific or bispecific antibodies, disulfide stabilized Fv fragments, scFv tandems, diabodies, tribodies or tetrabodies, peptoids, peptide or nucleic acid aptamers, antibody mimetics or combinations thereof. an increase or a decrease the antibody mimetics comprise DARPins, affibody molecules, affilins, affitins, anticalins, avimers, fynomers, Kunitz domain peptides or monobodies. an increase or a decrease the single chain antibodies or fragments comprise Fv fragments; single chain Fv (scFv) fragments, Fab′ fragments, F(ab′)₂ fragments, Fv, Fab′-SH, single-domain V_{H} single antibodies or combinations thereof. In certain embodiments, the immunomodulatory agent is a Toll-like receptor (TLR) agonist. In certain embodiments, a TLR comprises TLR2, TLR3, TLR4, TLR7, TLR8, TLR9 or combinations thereof. In certain embodiments, the TLR is TLR9. In certain embodiments, the TLR9 agonist comprises Poly-ICLC, LPS, MPL, CpG ODN or small molecules. In certain embodiments, the TLR agonist is conjugated to the CD47 binding domain via a linker or a covalent bond. In certain embodiments, the covalent bond is a disulfide bond or any environmentally sensitive bond. In certain embodiments, the method further comprising treatment with a secondary therapeutic agent. In certain embodiments, a secondary therapeutic agent comprises a chemotherapeutic agent, cytokines, an inhibitor of immune checkpoint suppressive activity, an agonist of immune stimulatory activity, a growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, radiotherapy, or combinations thereof.

In another aspect, a composition comprises an anti-CD47 binding domain conjugated to at least one Toll-like receptor (TLR) agonist. In certain embodiments, the TLR comprises TLR2, TLR3, TLR4, TLR7, TLR8, TLR9 or combinations thereof. In certain embodiments, the TLR is TLR9. In certain embodiments, the TLR9 agonist comprises Poly-ICLC, LPS, MPL, CpG ODN or small molecules. In certain embodiments, the TLR agonist is conjugated to the anti-CD47 binding domain via a linker or a covalent bond.

In certain embodiments, a TLR2 agonist comprises CU-T12-9, Pam3CSK4, FSL-1, Pam2CSK4, CL429 or combinations thereof.

In certain embodiments, a TLR3 agonist comprises: Poly(A: U), Poly(I: C) and derivatives/analogs thereof.

In certain embodiments, a TLR4 agonist comprises: Lipopolysaccharides (LPS), Monophosphoryl Lipid A and derivatives/analogs thereof.

In certain embodiments, a TLR5 agonist comprises Flagellins.

In certain embodiments, TLR7/8 agonists comprise Thiazoquinoline derivatives, imidazoquinoline derivatives, adenine analogs, guanosine analogs, thymidine analogs, benzoazepine analogs or combinations thereof. In certain embodiments, Thiazoquinolone derivatives include CL075. In certain embodiments, Imidazoquinoline derivatives comprise CL097, gardiquimod, imiquimod, resiquimod or combinations thereof.

In certain embodiments, a TLR9 agonist comprises CpG oligodeoxynucleotides (ODN). In certain embodiments, CpG ODNs comprise ODN 1585, ODN 2216, ODN 2336, ODN 1668, ODN 1826, ODN 2006, ODN 2007, ODN BW006, ODN D-SL01, ODN 2395, ODN M362, ODN D-SL03 or combinations thereof.

In another aspect, a composition comprises an anti-CD47 binding domain and one or more immunomodulatory agents. In certain embodiments, the one or more immunomodulatory agents modulates an innate immune response, an adaptive immune response or the combination thereof. In certain embodiments, the one or more immunomodulatory agents comprise a Toll-like receptor (TLR) agonist, Inducible T cell Co-stimulator (CD278), OX40 (CD134), 41 BB, Glucocorticoid-induced Tumor Necrosis Factor Receptor (GITR), CD40, CD27, stimulator of interferon (IFN) genes (STING) agonists or combinations thereof. Examples of STING agonists include DMXAA, ASA404, cyclic dinucleotides (CDNs), non-cyclic dinucleotides (non-CDNs) or combinations thereof. An example of a CDN is ADU-S100/MIW815 or MK-1454. An example of a non-CDN is MK-2118 or E7766.

### General Definitions

Unless specifically defined otherwise, all technical and scientific terms used herein shall be taken to have the same meaning as commonly understood by one of ordinary skill in the art (e.g., in cell culture, molecular genetics, and biochemistry).

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

As used herein, the term “about” in the context of a numerical value or range means ±10% of the numerical value or range recited or claimed, unless the context requires a more limited range.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

The term “adaptive immunity” as used herein, also known as acquired immunity or specific immunity, refers to the specific immunity of the body against the antigen formed after the stimulation of the antigen molecule, which involves cellular immunity and humoral immunity.

As used herein, the term “antibody” is used in reference to any immunoglobulin molecule that reacts with a specific antigen. It is intended that the term encompass any immunoglobulin (e.g., IgG, IgM, IgA, IgE, IgD, etc.) obtained from any source (e.g., humans, rodents, non-human primates, caprines, bovines, equines, ovines, etc.). Specific types/examples of antibodies include polyclonal, monoclonal, humanized, chimeric, human, or otherwise-human-suitable antibodies. “Antibodies” also includes derivatives or any functional (antigen-binding) antibody fragments, including fragment antigen binding (Fab) fragments, F(ab′)₂ fragments, Fab′ fragments, Fv fragments, recombinant IgG(rlgG) fragments, variable heavy chain (V_{H}) regions capable of specifically binding the antigen, single chain antibody fragments, including single chain variable fragments (scFv), and single domain antibodies (e.g., sdAb, sdFv, nanobody) fragments. The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof, IgM, IgE, IgA, and IgD.

As used herein “antibody-dependent cell-mediated cytotoxicity” or “ADCC” refers to a cell-mediated reaction in which nonspecific cytotoxic cells that express Fc receptors (FcRs) (e.g. natural killer (NK) cells, neutrophils, and macrophages) recognize bound antibody on a target cell and subsequently cause lysis of the target cell. ADCC activity of a molecule of interest can be assessed using an in vitro ADCC assay, such as that described in U.S. Pat. No. 5,500,362 or 5,821,337. Useful effector cells for such assays include peripheral blood mononuclear cells (PBMC) and NK cells. Alternatively, or additionally, ADCC activity of the molecule of interest may be assessed in vivo, e.g., in an animal model such as that disclosed in Clynes et al., 1998, PNAS (USA), 95:652-656.

As used herein, the term “antigen” is generally used in reference to any substance that is capable of reacting with an antibody. More specifically, as used herein, the term “antigen” refers to a synthetic peptide, polypeptide, protein or fragment of a polypeptide or protein, or any other molecule which elicits an antibody response in a subject or is recognized and bound by an antibody.

As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. In some embodiments, a sufficient number of weaker interactions can provide sufficient stability for moieties to remain physically associated under a variety of different conditions.

The term “biological sample” encompasses a variety of sample types obtained from an organism and can be used in a diagnostic or monitoring assay. The term encompasses blood and other liquid samples of biological origin, solid tissue samples, such as a biopsy specimen or tissue cultures or cells derived therefrom and the progeny thereof. The term encompasses samples that have been manipulated in any way after their procurement, such as by treatment with reagents, solubilization, or enrichment for certain components. The term encompasses a clinical sample, and also includes cells in cell culture, cell supernatants, cell lysates, serum, plasma, biological fluids, and tissue samples.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g., clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer. “Reducing growth of cancer cells” includes, but is not limited to, reducing proliferation of cancer cells, and reducing the incidence of a non-cancerous cell becoming a cancerous cell. Whether a reduction in cancer cell growth has been achieved can be readily determined using any known assay, including, but not limited to, [³H]-thymidine incorporation; counting cell number over a period of time; detecting and/or measuring a marker associated with the cancer, etc.

The term “checkpoint inhibitor” means a group of molecules on the cell surface of CD4⁺ and/or CD8⁺ T cells that fine-tune immune responses by down-modulating or inhibiting an anti-tumor immune response. “Anti-immune checkpoint inhibitor therapy” refers to the use of agents that inhibit immune checkpoint inhibitors. Inhibition of one or more immune checkpoint inhibitors can block or otherwise neutralize inhibitory signaling to thereby upregulate an immune response in order to more efficaciously treat cancer, or any infectious disease.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

An “environmentally sensitive bond” refers to a bond that can be broken based on its properties. For example, the bond may be a peptide bond and it is cleavable by a peptidase.

A “host cell”, as used herein, refers to a microorganism or a eukaryotic cell or cell line cultured as a unicellular entity which can be, or has been, used as a recipient for a recombinant vector or other transfer polynucleotides, and include the progeny of the original cell which has been transfected. It is understood that the progeny of a single cell may not necessarily be completely identical in morphology or in genomic or total DNA complement as the original parent, due to natural, accidental, or deliberate mutation.

The term “immune response” refers to any detectable response to a particular substance (such as an antigen or immunogen) by the immune system of a host mammal, such as innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids).

The term “immunogenic” refers to the ability of a substance to cause, elicit, stimulate, or induce an immune response, or to improve, enhance, increase or prolong a pre-existing immune response, against a particular antigen, whether alone or when linked to a carrier, in the presence or absence of an adjuvant.

The term “immunomodulatory agent” as used herein refers to the ability of a substance to increase or a decrease an immune response to an antigen as compared to a control. Examples of immunomodulatory agents include without limitation an immune checkpoint molecule, a monoclonal antibody, an antibody fragment, an aptamer, an siRNA, an antisense molecule, a fusion protein, a cytokine, a cytokine receptor, a bronchodilator, a statin, an anti-inflammatory agent (e.g. methotrexate), an NSAID, TLR agonists, stimulator of interferon (IFN) genes (STING) agonists or combinations thereof. Examples of STING agonists include DMXAA, ASA404, cyclic dinucleotides (CDNs), non-cyclic dinucleotides (non-CDNs) or combinations thereof. An example of a CDN is ADU-S100/MIW815 or MK-1454. An example of a non-CDN is MK-2118, TTI-10001, ALG-031048 or E7766 and the like.

The term “innate immunity” as used herein refers to the natural immune defense function formed during germline development and evolution, that is, the non-specific defense function already possessed after birth, also known as non-specific immunity. Innate immunity involves a variety of cells and molecules, such as macrophages, natural killer cells, complement, cytokines (IL, CSF, IFN, TNF, TGFβ), chemokines (including CC chemokines, such as CCL1, CCL2), CCL3, CCL4, CCL5, CCL6, CCL7, CCL8, CCL9, CCL10, CCL11, CCL12, etc. CXC chemokines, such as CXCL1, CXCL2, CXCL3, CXCL4, CXCL5, CXCL6, CXCL7, CXCL8, CXCL9, etc., C chemokine, CX3C chemokine), lysozyme and so on.

As used throughout, “modulation” is meant to refer to an increase or a decrease in the indicated phenomenon (e.g., modulation of a biological activity refers to an increase in a biological activity or a decrease in a biological activity).

The terms “phagocytic cells” and “phagocytes” are used interchangeably herein to refer to a cell that is capable of phagocytosis. There are three main categories of phagocytes: macrophages, mononuclear cells (histiocytes and monocytes); polymorphonuclear leukocytes (neutrophils) and dendritic cells. There are several categories of phagocytes: macrophages; mononuclear cells (histiocytes and monocytes); polymorphonuclear leukocytes; (neutrophils) and dendritic cells. Macrophages are of particular interest. Phagocytosis-associated cell responses include immunomodulatory responses like the generation and release of pro-inflammatory and anti-inflammatory mediators, and also cell responses of destructive nature such as the respiratory burst, and the release of toxic and microbicidal molecules by degranulation. Professional phagocytes are capable of recognizing a wide variety of phagocytic targets, and of ingesting them at a higher rate than non-phagocytic cells.

Neutrophils and macrophages are representative of fully differentiated phagocytes. While neutrophils leaving the bone marrow are fully differentiated, macrophages differentiate from circulating monocytes in extra-vascular tissues. Monocytes display a lower phagocytic response, compared to neutrophils and macrophages, and must respond to activation and differentiation signals in order to achieve optimal phagocytic capacity. The process of monocyte-to-macrophage differentiation has been well characterized and can be performed in vitro or in vivo.

A “therapeutically effective dose” or “therapeutic dose” is an amount sufficient to effect desired clinical results (i.e., achieve therapeutic efficacy). For purposes of this disclosure, a therapeutically effective dose of anti-CD47 chimeric molecule is an amount that is sufficient to palliate, ameliorate, stabilize, reverse, prevent, slow or delay the progression of the disease state (e.g., cancer or chronic infection) by inducing an immune response, e.g. macrophage mediated killing of a target cell. Thus, a therapeutically effective dose of an anti-CD47 chimeric molecule can decrease the target cell population through an in vivo immune response by at least about 10%, at least about 20%, at least about 50%, at least about 75%, at least about 90% or more, relative to the effect in the absence of administering an anti-CD47 chimeric molecule.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder, or disease, slowing the progression of the condition, disorder or disease temporarily, although in some instances, it involves halting the progression of the condition, disorder or disease permanently.

As used herein, the terms “treat” and “prevent” are not intended to be absolute terms. In various embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus, the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The term “variable region” or “variable domain”, when used in reference to an antibody, such as an antibody fragment, refers to the domain of an antibody heavy or light chain that is involved in binding the antibody to antigen. The variable domains of the heavy chain and light chain (V_{H} and V_{L}, respectively) of a native antibody generally have similar structures, with each domain comprising four conserved framework regions (FRs) and three CDRs. (See, e.g., Kindt et al. Kuby Immunology, 6th ed., W. H. Freeman and Co., page 91 (2007). A single V_{H} or V_{L} domain may be sufficient to confer antigen-binding specificity. Furthermore, antibodies that bind a particular antigen may be isolated using a V_{H} or V_{L} domain from an antibody that binds the antigen to screen a library of complementary V_{L} or V_{H} domains, respectively. See, e.g., Portolano et al., J. Immunol. 150:880-887 (1993); Clarkson et al., Nature 352:624-628 (1991).

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The practice of the present disclosure employs, unless otherwise indicated, conventional techniques of chemistry, molecular biology, microbiology, recombinant DNA, genetics, immunology, cell biology, cell culture and transgenic biology, which are within the skill of the art. See, e.g., Maniatis et al., 1982, Molecular Cloning (Cold Spring Harbor Laboratory Press, Cold Spring Harbor, N.Y.); Sambrook et al., 1989, Molecular Cloning, 2nd Ed. (Cold Spring Harbor Laboratory Press, Cold Spring Harbor, N.Y.); Sambrook and Russell, 2001, Molecular Cloning, 3rd Ed. (Cold Spring Harbor Laboratory Press, Cold Spring Harbor, N.Y.); Ausubel et al., 1992), Current Protocols in Molecular Biology (John Wiley & Sons, including periodic updates); Glover, 1985, DNA Cloning (IRL Press, Oxford); Anand, 1992; Guthrie and Fink, 1991; Harlow and Lane, 1988, Antibodies, (Cold Spring Harbor Laboratory Press, Cold Spring Harbor, N.Y.); Jakoby and Pastan, 1979; Nucleic Acid Hybridization (B. D. Hames & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu et al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Riott, Essential Immunology, 6th Edition, Blackwell Scientific Publications, Oxford, 1988; Hogan et al., Manipulating the Mouse Embryo, (Cold Spring Harbor Laboratory Press, Cold Spring Harbor, N.Y., 1986); Westerfield, M., The zebrafish book. A guide for the laboratory use of zebrafish (Danio rerio), (4th Ed., Univ. of Oregon Press, Eugene, 2000).

Any compositions or methods provided herein can be combined with one or more of any of the other compositions and methods provided herein.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is a schematic representation showing a CD47-binding immune stimulating antibody conjugate (ISAC; αCD47-CpG hereafter).

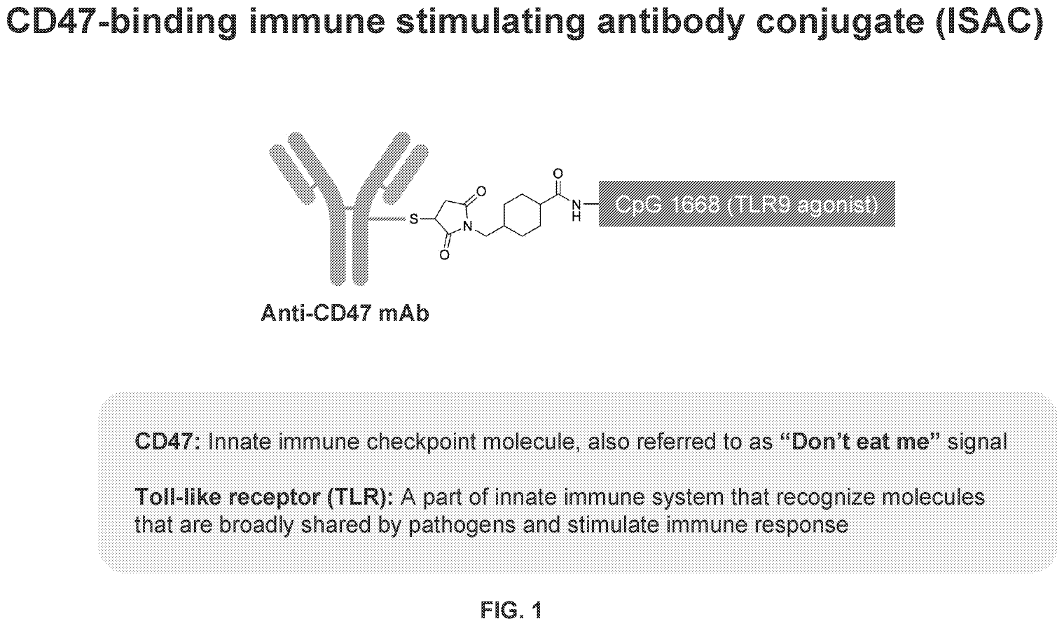

FIG. 2 is a schematic representation showing a synthesis scheme of αCD47-CpG conjugate.

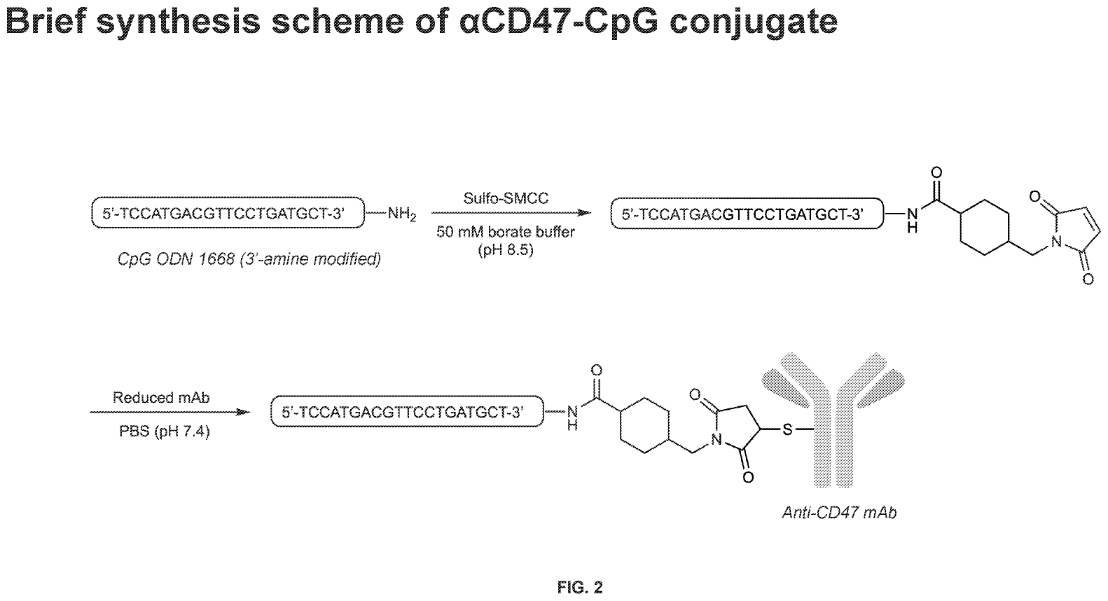

FIG. 3 is a schematic representation showing the pathways affected by the Toll-like receptor (TLR).

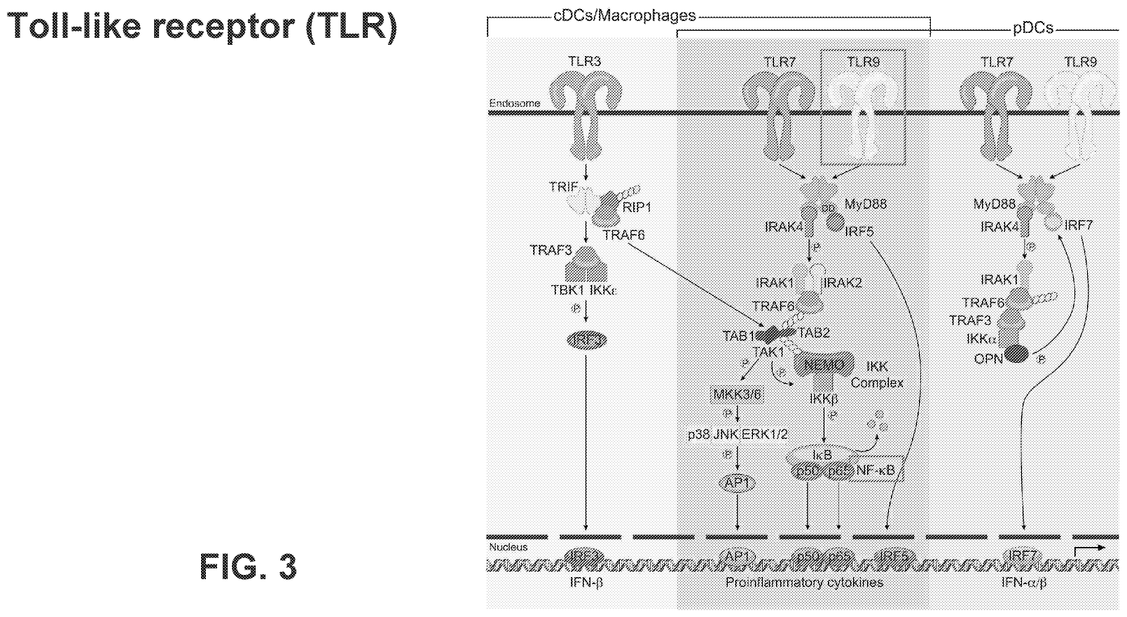

FIG. 4 is a schematic representation showing the hypothetical mechanism of αCD47-CpG.

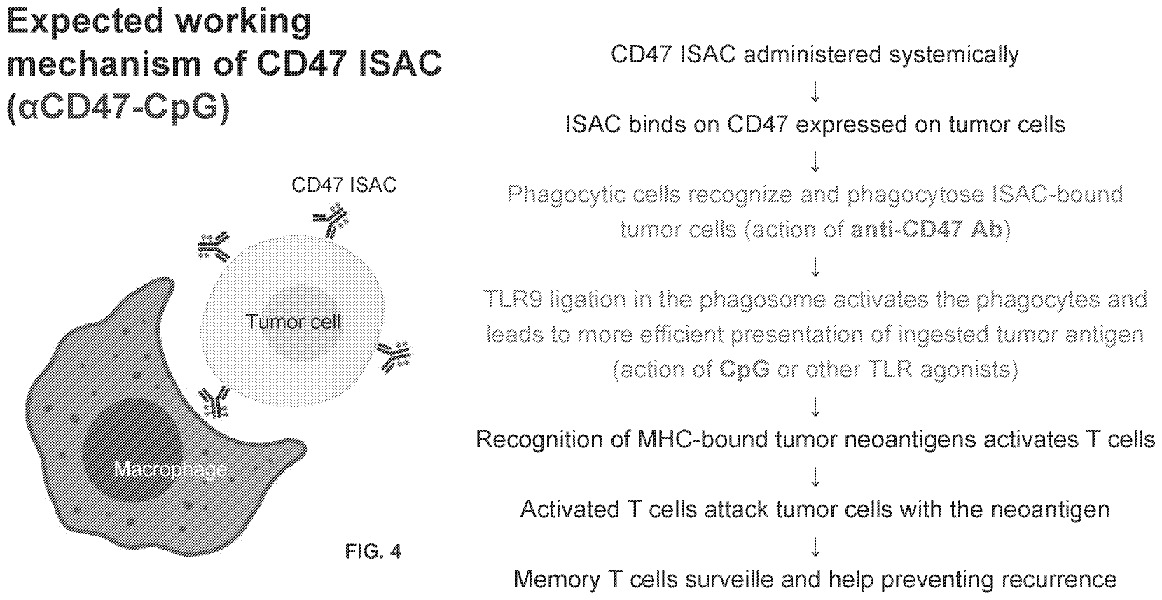

FIG. 5 is a blot and a graph showing the characterization of αCD47-CpG C.

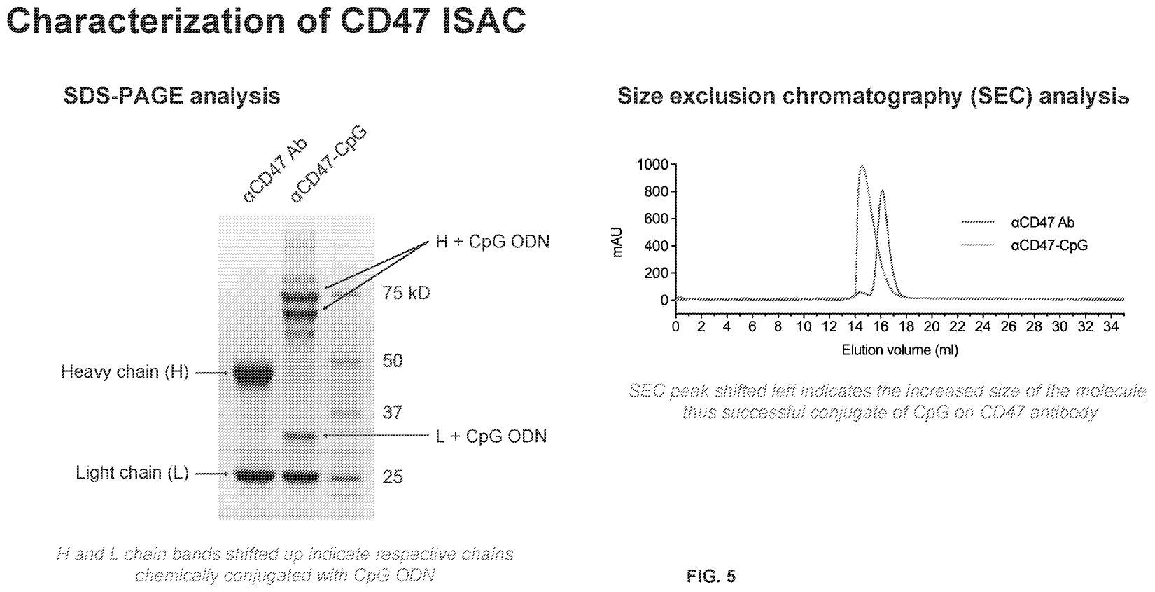

FIG. 6 is a series of blots showing the synthesis of αCD47-CpG with different DAR.

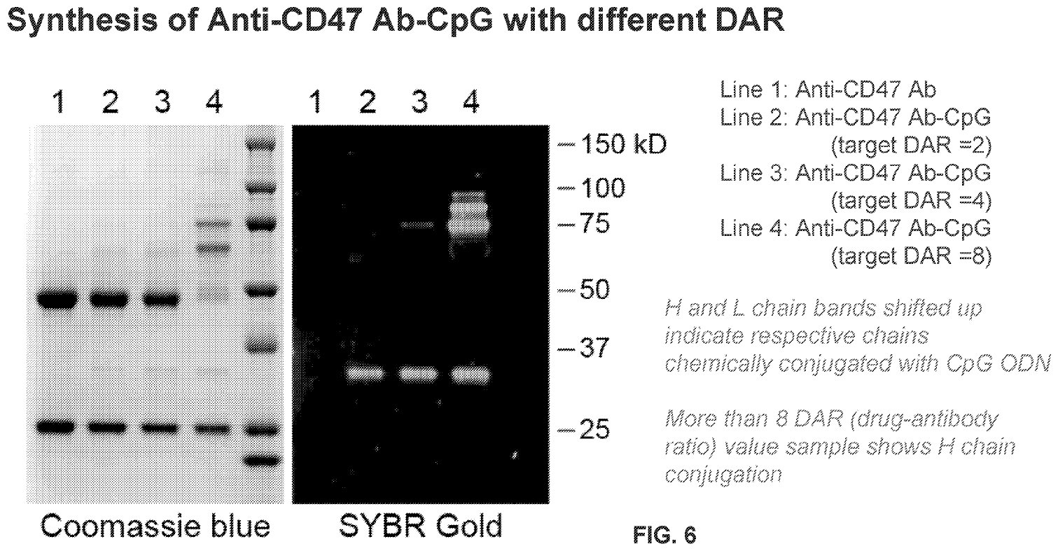

FIGS. 7A and 7B are graphs showing that the &CD47-CpG, unlike CpG, binds to CD47 in a dose-dependent manner. Binding curves of αCD47-CpG to (FIG. 7A) human or (FIG. 7B) mouse CD47.

FIG. 8 is a graph showing αCD47-CpG binding to TLR9 in a dose-dependent manner.

FIG. 9 is a series of graphs showing αCD47-CpG-mediated NF-κB activation in macrophages under monoculture or co-culture with tumor cells.

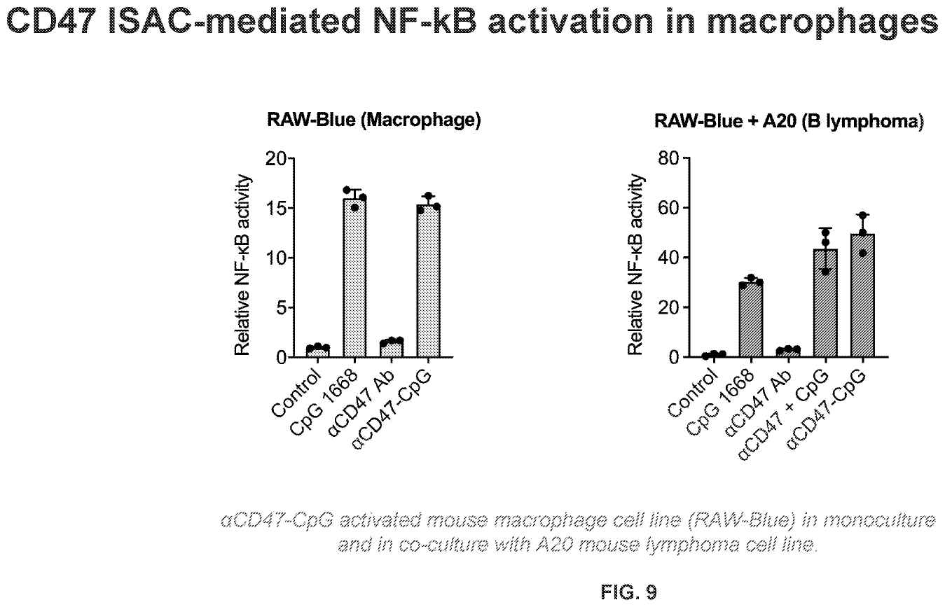

FIG. 10 is a graph showing results from a confocal microscopy-based phagocytosis assay where αCD47-CpG promotes phagocytosis of tumor cells.

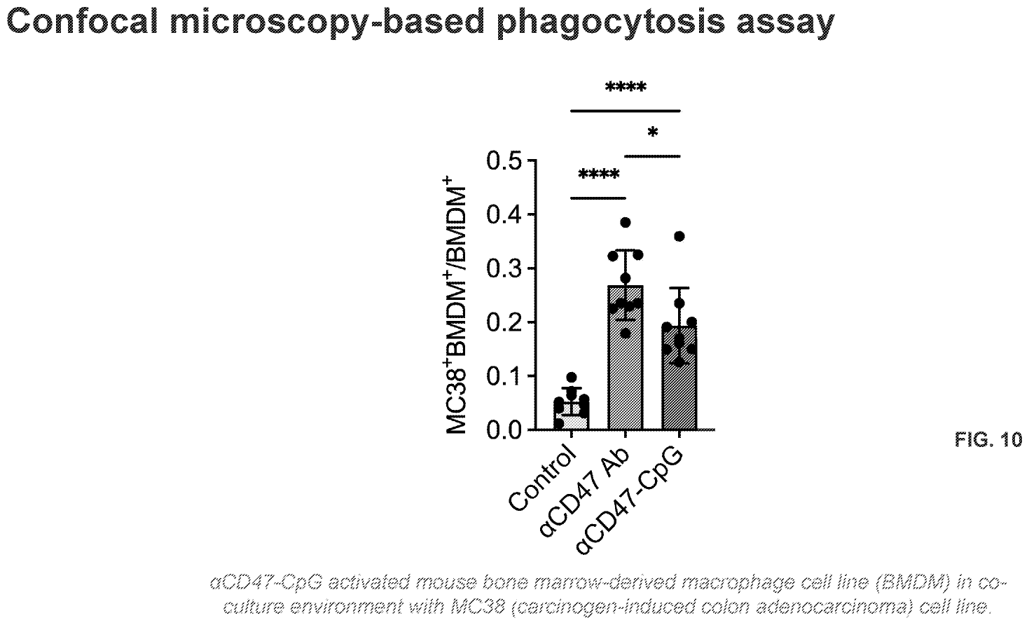

FIG. 11 is a series of cellular stains and a schematic showing the αCD47-CpG treatment on macrophage and tumor cell co-culture model.

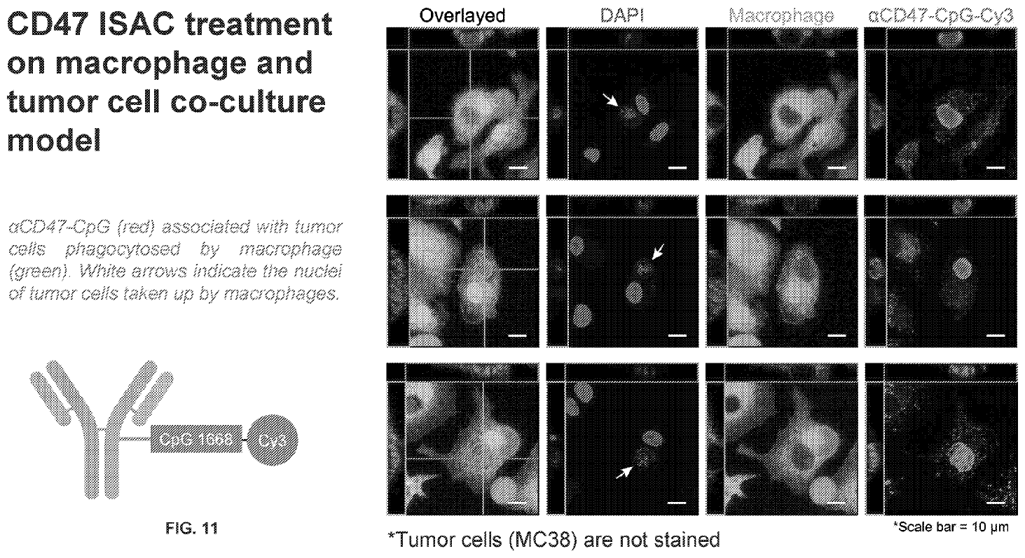

FIG. 12 is a series of cellular stains and a schematic showing αCD47-CpG treatment on macrophage and tumor cell co-culture model (zoomed-in).

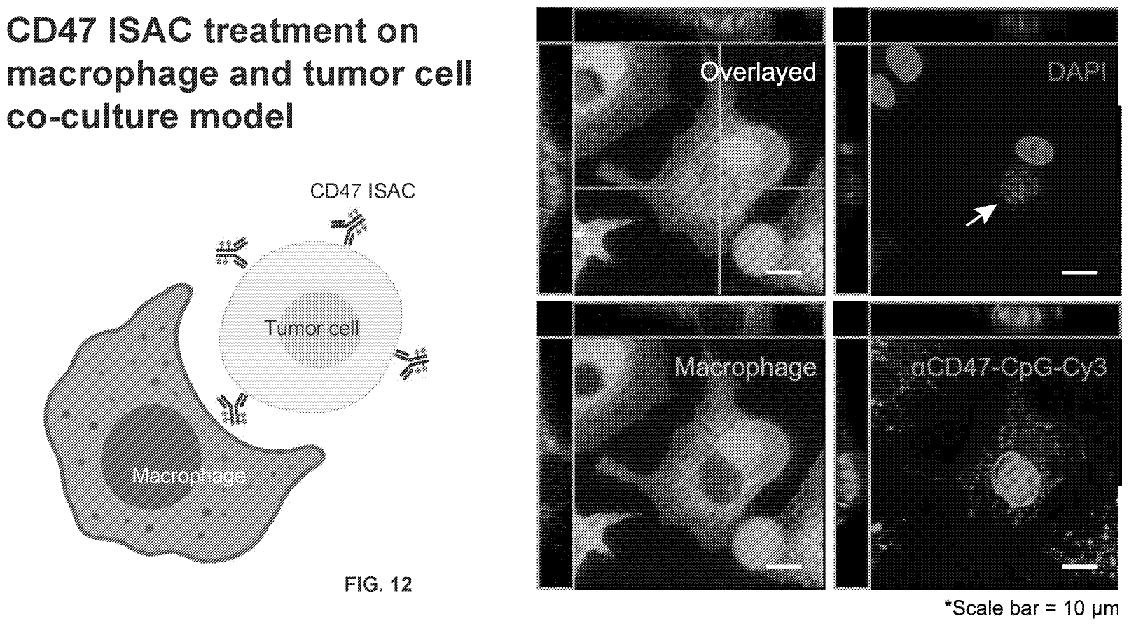

FIG. 13 is a series of graphs showing results from a BMDM+MC38 Cytokine induction assay (ELISA) where αCD47-CpG induces the release of pro-inflammatory cytokines.

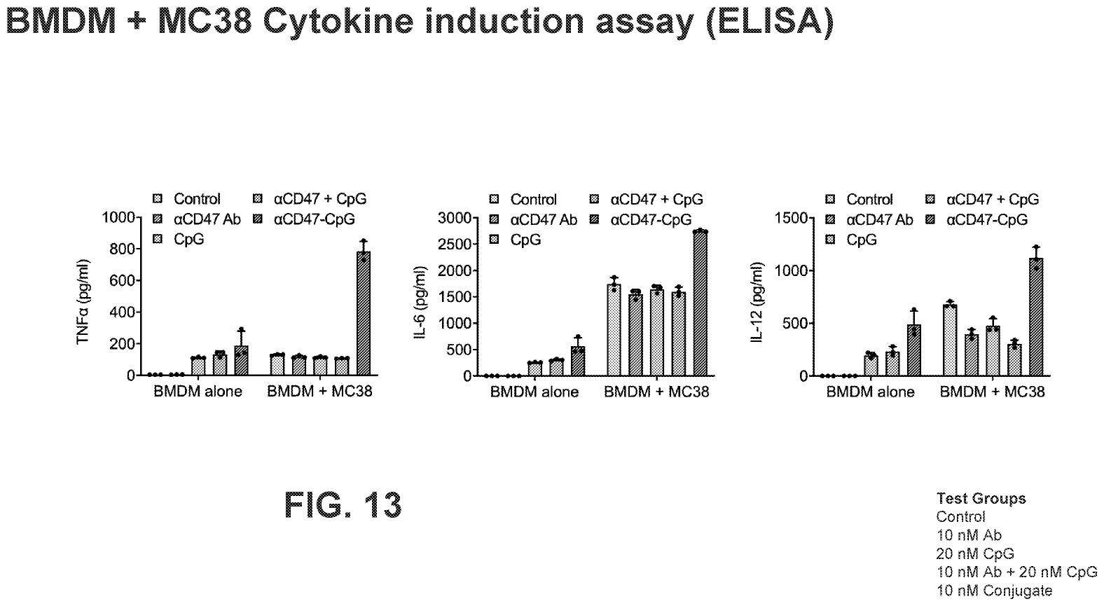

FIG. 14 is a series of stains showing results from an in vivo biodistribution study where αCD47-CpG enhances the systemic delivery of CpG to the tumor tissue (in comparison to CpG).

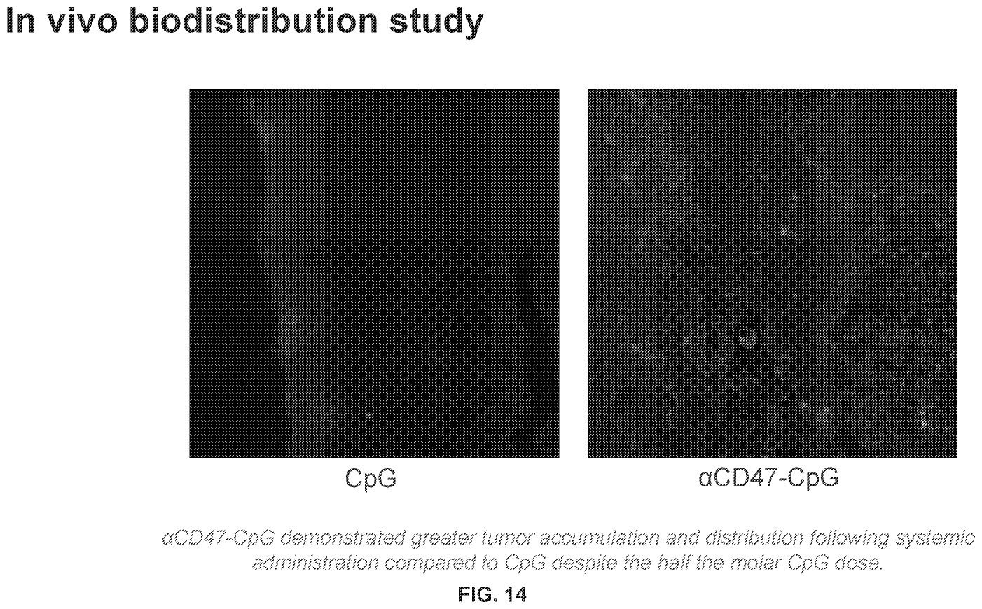

FIG. 15 is a schematic and a graph showing results of the in vivo efficacy of αCD47-CpG on MC38 model.

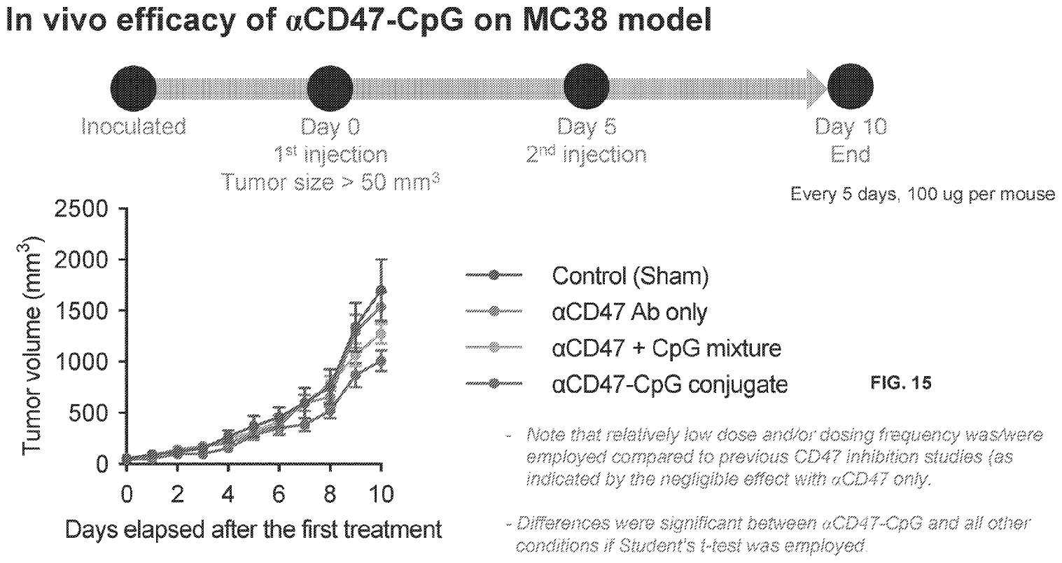

FIGS. 16A-16C are a series of graphs and confocal micrographs showing that αCD47-CpG promotes macrophage uptake of cancer cells in vitro and suppresses tumor growth in vivo. (FIG. 16A) Representative confocal micrograph showing phagocytosis (white arrow) of αCD47-CpG (red) accumulated M38 mouse colon adenocarcinoma cells by macrophages (green) (DAPI: blue; Scale bar=10 μm) and (FIG. 16B) quantitative analysis. (FIG. 16C) In vivo anti-cancer effect of αCD47-CpG against M38-based flank tumor. * P<0.05.

### DETAILED DESCRIPTION

Human CD47 (UniProtKB—Q08722 (CD47_HUMAN; IAP)) is a transmembrane protein that binds the ligands thrombospondin-1 (TSP-1) and signal-regulatory protein alpha (SIRPα; CD172a; UniProtKB P78324) and can act as a “don't eat me” signal to the immune system, especially for macrophages which express SIRPα. Potent inhibition (low IC₅₀) of the binding of SIRPα to CD47 on the surface of tumor cells is a measure to increase the phagocytosis of tumor cells by macrophages. CD47 is involved in a range of cellular processes, including apoptosis, proliferation, adhesion, and migration. Furthermore, it plays a key role in immune and angiogenic responses. CD47 is overexpressed in tumor cells from patients with both hematological and solid tumors. Antibodies against CD47 are described in the state of the art and have shown promising preclinical and early clinical activity in different tumor entities, including hematological malignancies such as lymphoma and solid tumors, for example gastric cancer (Weiskopf K., European Journal of Cancer 76 (2017) 100-109; Huang Y et al., J Thorac Dis 2017; 9 (2): E168-E174; Kaur et al., Antibody Therapeutics, 3 (2020) 179-192). Antibodies of the IgG1 subclass that bind CD47 can result in the depletion of platelets and reduction of red blood cells (RBC) and hemoglobin in a Fc-dependent manner (see e.g., US20140140989). For avoiding this adverse effect, in WO2017196793 there is described a mutant form of the IgG4 subclass of an anti-CD47 antibody (IgG4PE, with the S228P mutation as well as a L235E mutation to reduce FcTR binding). Such an anti-CD47 antibody with severely reduced FcTR binding and effector function does not result in such platelet depletion.

### Anti-CD47-Toll-like Receptor (TLR) Agonist Chimeric Molecule

The disclosure herein, provides for a chimeric molecule wherein the anti-CD47 molecule is conjugated to a TLR agonist and overcomes any deleterious effects in vivo associated with Fc mediated depletion of platelets and red blood cells. The chimeric molecule either lacks an Fc receptor, as in the case of an scFV, or the conjugation to a TLR agonist masks the Fc receptor or is inaccessible.

Accordingly, in certain embodiments, a composition comprises an anti-CD47 agent conjugated to a toll-like receptor (TLR) agonist.

In some embodiments, a subject anti-CD47 agent is an antibody that specifically binds CD47 (i.e., an anti-CD47 antibody) and reduces the interaction between CD47 on one cell (e.g., a tumor cell) and SIRPα on another cell (e.g., a phagocytic cell). In some embodiments, a suitable anti-CD47 antibody does not activate CD47 upon binding. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized chimeric versions of such antibodies. Humanized antibodies (e.g., hu5F9-G4) are especially useful for in vivo applications in humans due to their low antigenicity. Similarly, caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In one embodiment, the anti-CD47 agent comprises: polypeptides such as antibodies or antibody fragments bearing epitope recognition sites, such as Fab, Fab′, F(ab′)₂ fragments, Fv fragments, single chain antibodies, single chain Fv (scFv) fragments; Fab′ fragments; F(ab′)₂ fragments, antibody mimetics (such as DARPins, affibody molecules, affilins, affitins, anticalins, avimers, fynomers, VHH antibodies (nanobody), humanized antibodies and antibody fragments; camelized antibodies and antibody fragments, human antibodies and antibody fragments, monospecific or bispecific antibodies, diabodies, tribodies or tetrabodies, peptoids, peptide or nucleic acid aptamers, Kunitz domain peptides and monobodies), recombinant probes, peptoids, aptamers and the like.

Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody as well as production by recombinant host cells. In some embodiments, the antibodies are recombinantly-produced fragments, such as fragments comprising arrangements that do not occur naturally, such as those with two or more antibody regions or chains joined by synthetic linkers, e.g., peptide linkers. In some aspects, the antibody fragments are scFvs.

In some embodiments, the anti-CD47 antibody comprises a bispecific antibody or binding fragment thereof. In some embodiments, the bispecific antibody or binding fragment thereof is a bispecific antibody conjugate, a hybrid bispecific IgG, a variable domain only bispecific antibody, a CH1/CL fusion protein, a Fab fusion protein, a non-immunoglobulin fusion protein, a Fc-modified IgG, an appended & Fc-modified IgG, a modified Fc and CH3 fusion protein, an appended IgG-HC fusion, a Fc fusion, a CH3 fusion, an IgE/IgM CH2 fusion, or a F(ab′)₂ fusion.

In some embodiments, a bispecific antibody or binding fragment includes a Knobs-into-Holes (KiH), Asymmetric Re-egnineering Technology-immunoglobulin (ART-Ig), Triomab quadroma, bispecific monoclonal antibody (BiMAb, BsmAb, BsAb, bsMab, BS-Mab, or BiMAb), FcAAdp, XmAb, Azymetric, Bispecific Engagement by Antibodies based on the T-cell receptor (BEAT), Bispecific T-cell Engager (BiTE), Biclonics, Fab-scFv-Fc, Two-in-one/Dual Action Fab (DAF), FinomAb, scFv-Fc-(Fab)-fusion, Dock-aNd-Lock (DNL), Adaptir (previously SCORPION), Tandem diAbody (TandAb), Dual-affinity-ReTargeting (DART), or nanobody.

In some embodiments, a variable domain only bispecific antibody comprises a tandem scFv (taFv), triplebody, diabody (db), dsDb, db (KiH), scDb, dsFv-dsFv′, t and Abs, triple heads, tandem dAb/VHH, triple dAb/VHH, or tetravalent dAb/VHH.

In some embodiments, a CH1/CL fusion protein comprises a scFv2-CH1/CL or VHH2-CH1/CL.

In some embodiments, Fab fusion protein comprises a Fab-scFv (bibody), Fab-scFv2 (tribody), Fab-Fv, Fab-dsFv, Fab-VHH, or orthogonal Fab-Fab.

In some embodiments, a non-immunoglobulin fusion protein comprises a scFv2-albumin, scDb-albumin, taFv-albumin, taFv-toxin, miniantibody, DNL-Fab2, DNL-Fab2-scFv, DNL-Fab2-IgG-cytokine2, or ImmTAC (TCR-scFv).

In some embodiments, a Fc-modified IgG comprises a IgG(KiH), IgG(KiH) common LC, ZW1 IgG common LC, Biclonics common LC, CrossMAb, scFab-IgG(KiH), Fab-scFab-IgG(KiH), orthogonal Fab IgG(KiH), DuetMab, CH3 charge pair+CH1/CL charge pair, hinge/CH3 charge pair, DuoBody, four-in-one-CrossMab (KiH), LUZ-Y common LC, LUZ-Y scFab-IgG, or FcFc*.

In some embodiments, an appended & Fc-modified IgG comprises an IgG(KiH)-Fv, IgG(HA-TF-FV), IgG(KiH)-scFab, scFab-Fc (KiH)-scFv2, scFab-Fc (KiH)-scFv, half DVD-Ig, Dual Variable Domain-immunoglobulin (DVD-Ig), or CrossMab-Fab.

In some embodiments, a modified Fc and CH3 fusion protein comprises a scFv-Fc (KiH), scFv-Fc (CH3 charge pair), scFv-FC (EW-RVT), scFv-Fc (HA-TF), scFv-Fc (SEEDbody), taFv-Fc (KiH), scFv-Fc (KiH)-Fv, Fab-Fc (KiH)-scFv, Fab-scFv-Fc (KiH), Fab-scFv-Fc (BEAT), DART-Fc, scFv-CH3 (KiH), or TriFabs.

In some embodiments, an appended IgG-HC fusion antibody comprises IgG-HC-scFv, IgG-dAb, IgG-taFv, IgG-CrossFab, IgG-orthogonal Fab, IgG-(CaCO) Fab, scFv-HC-IgG, tandem Fab-IgG, Fab-IgG(CaCpFab), Fab-IgG(CR3), or Fab-hinge-IgG(CR3).

In some embodiments, an appended IgG-LC fusion antibody comprises IgG-scFv (LC), scFv (LC)-IgG, or dAb-IgG.

In some embodiments, an anti-CD47 antibody described herein comprises a scFv that is further conjugated to an additional polypeptide comprising a hinge region, a transmembrane domain, a co-stimulatory domain (e.g., CD28, 4-1BB, CD27, or others) and a CD3 (activation domain to generate a chimeric antigen receptor (CAR) expressed on a T cell.

In some embodiments, an anti-CD47 antibody described herein comprises an IgG framework, an IgA framework, an IgE framework, or an IgM framework. In some embodiments, the anti-CD47 antibody comprises an IgG framework (e.g., IgG1, IgG2, IgG3, or IgG4). In such embodiments, the anti-CD47 antibody comprises an IgG1, IgG2, IgG3, or an IgG4 framework.

In some embodiments, the anti-CD47 antibody further comprises a mutation in a framework region. In some embodiments, the mutation is in the CH2 or CH3 region. In other embodiments, the mutation is in the hinge region. In some embodiments, the mutation is to stabilize the antibody and/or to increase half-life. In some embodiments, the mutation is to modulate Fc receptor interactions, e.g., to reduce or eliminate Fc effector functions such as FcγR, antibody-dependent cell-mediated cytotoxicity (ADCC) or complement-dependent cytotoxicity (CDC). In additional embodiments, the mutation is to modulate glycosylation.

In some embodiments, the anti-CD47 antibody comprises an IgG1 framework. In some embodiments, the constant region of the anti-CD47 antibody is modified at one or more amino acid positions to alter Fc receptor interaction. In some embodiments, the one or more amino acid positions comprise E233, L234, L235, G236, T250, M252, S254, T256, K322, A327, A330, P331, M428, H433, or N434 (Kabat numbering; EU index of Kabat et al 1991 Sequences of Proteins of Immunological Interest). In some embodiments, the mutation comprises E233P, L234A, L234V, L235E, L235A, G236A, T250Q, M252Y, S254T, T256E, K322A, A327G, A330S, P331S, M428L, H433K, or N434F.

In some embodiments, the modification at one or more amino acid positions in the IgG1 constant region to alter Fc receptor interaction leads to increased half-life. In some embodiments, the modification at one or more amino acid positions comprises T250, M252, S254, T256, M428, H433, N434, or a combination thereof, e.g., comprising T250Q/M428L or M252Y/S254T/T256E and H433K/N434F.

In some embodiments, the modification at one or more amino acid positions in the IgG1 constant region to alter Fc receptor interaction leads to reduced ADCC and/or CDC. In some embodiments, the modification at one or more amino acid positions comprises E233, L234, L235, G236, A327, K322, A330, P331, or a combination thereof. In some embodiments, the modification at one or more amino acid positions for reduced ADCC and CDC comprises, e.g., the combination E233P/L234V/L235A/G236 and A327G/A330S/P331S. In some embodiments, the modification at one or more amino acid positions for reduced CDC comprises K332A.

In some embodiments, the modification at one or more amino acid positions in the IgG1 constant region to alter Fc receptor interaction leads to increased macrophage phagocytosis. In some embodiments, the modification at one or more amino acid positions comprises G236, S239, 1332, or a combination thereof. In some embodiments, the modification at one or more amino acid positions for increased macrophage phagocytosis comprises the combination S239D/1332I/G236A.

In some embodiments, the human IgG constant region is modified to alter antibody-dependent cellular cytotoxicity (ADCC) and/or complement-dependent cytotoxicity (CDC), e.g., with a amino acid modification described in Natsume et al., 2008 Cancer Res, 68 (10): 3863-72; Idusogie et al., 2001 J Immunol, 166 (4): 2571-5; Moore et al., 2010 mAbs, 2 (2): 181-189; Lazar et al., 2006 PNAS, 103 (11): 4005-4010, Shields et al., 2001 JBC, 276 (9): 6591-6604; Stavenhagen et al., 2007 Cancer Res, 67 (18): 8882-8890; Stavenhagen et al., 2008 Advan. Enzyme Regul., 48:152-164; Alegre et al, 1992 J Immunol, 148:3461-3468; Reviewed in Kaneko and Niwa, 2011 Biodrugs, 25 (1): 1-11.

In some embodiments, the human IgG constant region is modified to induce heterodimerization. For example, having an amino acid modification within the CH3 domain at Thr366, which when replaced with a more bulky amino acid, e.g., Trp (T366W), is able to preferentially pair with a second CH3 domain having amino acid modifications to less bulky amino acids at positions Thr366, Leu368, and Tyr407, e.g., Ser, Ala and Val, respectively (T366S/L368A/Y407V). In some embodiments, heterodimerization via CH3 modifications is further stabilized by the introduction of a disulfide bond, for example by changing Ser354 to Cys (S354C) and Y349 to Cys (Y349C) on opposite CH3 domains (Reviewed in Carter, 2001 Journal of Immunological Methods, 248:7-15).

In some embodiments, an anti-CD47 antibody described herein lacks glycosylation, but is not modified at amino acid Asn297 (Kabat numbering). In these embodiments, the glycosylation is, for example, eliminated by production of the antibody in a host cell that lacks a post-translational glycosylation capacity, for example a bacterial or yeast derived system or a modified mammalian cell expression system. In certain aspects, such a system is a cell-free expression system.

In certain embodiments, the bispecific antibody comprises an antigen binding domain directed against CD47 and the other antigen binding domain is directed against SIRPα. SIRPα, also known as Src homology 2 domain-containing protein tyrosine phosphatase substrate 1/brain Ig-like molecule with tyrosine-based activation motif/cluster of differentiation antigen-like molecule with tyrosine-based activation motif/cluster of differentiation antigen-like family member A (SHPS-1/BIT/CD172), is also a member of the immunoglobulin superfamily and is abundantly expressed in myeloid-lineage hematopoietic cells such as macrophages and dendritic cells. Interaction of CD47 with SIRPPα results in phosphorylation of the cytoplasmic immunoreceptor tyrosine-based inhibition (ITIM) motif on SIRPα, leading to recruitment of SUP-1 and SHP-2 phosphatases. The subsequent downstream effect is inhibition of myosin-IIA accumulation at the phagocytic synapse and thereby the inhibition of phagocytosis. As such, CD47 is considered as a negative immune checkpoint that sends a “don't eat me” signal by cells to prevent phagocytosis.

In certain embodiments, the bispecific anti-CD47 comprises a binding domain which specifically binds to a checkpoint inhibitor. Examples of immune checkpoint proteins are well known in the art and include, without limitation, CTLA-4, PD-1, VISTA, B7-H2, B7-H3, PD-L1, B7-H4, B7-H6, 2B4, ICOS, HVEM, PD-L2, CD160, gp49B, PIR-B, KIR family receptors, TIM-1, TIM-3, TIM-4, LAG-3, BTLA, SIRPα (CD47), CD48, 2B4 (CD244), B7.1, B7.2, ILT-2, ILT-4, TIGIT, and A2aR (see, for example, WO 2012/177624).

Exemplary agents useful for inhibiting immune checkpoint inhibitors include antibodies, small molecules, peptides, peptidomimetics, natural ligands, and derivatives of natural ligands, that can either bind and/or inactivate or inhibit immune checkpoint proteins, or fragments thereof, as well as RNA interference, antisense, nucleic acid aptamers, etc. that can downregulate the expression and/or activity of immune checkpoint inhibitor nucleic acids, or fragments thereof. Exemplary agents for upregulating an immune response include antibodies against one or more immune checkpoint inhibitor proteins block the interaction between the proteins and its natural receptor(s); a non-activating form of one or more immune checkpoint inhibitor proteins (e.g., a dominant negative polypeptide); small molecules or peptides that block the interaction between one or more immune checkpoint inhibitor proteins and its natural receptor(s); fusion proteins (e.g. the extracellular portion of an immune checkpoint inhibition protein fused to the Fe portion of an antibody or immunoglobulin) that bind to its natural receptor(s); nucleic acid molecules that block immune checkpoint inhibitor nucleic acid transcription or translation; and the like. Such agents can directly block the interaction between the one or more immune checkpoint inhibitors and its natural receptor(s) (e.g., antibodies) to prevent inhibitory signaling and upregulate an immune response. Alternatively, agents can indirectly block the interaction between one or more immune checkpoint proteins and its natural receptor(s) to prevent inhibitory signaling and upregulate an immune response. For example, a soluble version of an immune checkpoint protein ligand such as a stabilized extracellular domain can binding to its receptor to indirectly reduce the effective concentration of the receptor to bind to an appropriate ligand. In one embodiment, anti-PD-1 antibodies, anti-PD-L1 antibodies, and anti-CTLA-4 antibodies, either alone or used in combination.

In certain embodiments, the bispecific agent comprises a binding domain which specifically binds and activates innate and/or adaptive immunity. Examples include a co-stimulatory receptor agonist, a stimulator of innate immune cells, or an activator of innate immunity. The co-stimulatory receptor agonist may be an anti-OX40 antibody (e.g., MEDI6469, MEDI6383, MEDI0562, and MOXR0916), anti-GITR antibody (e.g., TRX518, and MK-4166), anti-CD137 antibody (e.g., Urelumab, and PF-05082566), anti-CD40 antibody (e.g., CP-870,893, and Chi Lob 7/4), or an anti-CD27 antibody (e.g., Varlilumab, also known as CDX-1127). The stimulators of innate immune cells include, but are not limited to, a KIR monoclonal antibody (e.g., lirilumab), an inhibitor of a cytotoxicity-inhibiting receptor (e.g., NKG2A, also known as KLRC and as CD94, such as the monoclonal antibody monalizumab, and anti-CD96, also known as TACTILE), and a toll like receptor (TLR) agonist. The TLR agonist may be BCG, a TLR7 agonist (e.g., polyOICLC, and imiquimod), a TLR8 agonist (e.g., resiquimod). The activators of innate immune cells, such as natural killer (NK) cells, macrophages, and dendritic cells, include IDO inhibitors, TGFβ inhibitor, IL-10 inhibitor. An exemplary activator of innate immunity is Indoximod. Another exemplary activator is a stimulator of interferon genes (STING) agonist. In some embodiments, exemplary immunostimulatory cytokines include, but are not limited to, GM-CSF, G-CSF, IFNγ, IFNα; IL-2 (e.g., denileukin difitox), IL-6, IL-7, IL-10, IL-11, IL-12, IL-15, IL-18, IL-21, and TNFα. In some embodiments, the cytokines are pegylated (e.g., pegylated IL-2, IL-10, IFNγ, and IFNα).

In certain embodiments, a bispecific antibody comprises an antigen binding domain directed against CD47 and the other antigen binding domain is directed against a cancer cell marker, such as EGFR; HER2; CD96, CD97, CD99, PTHR2, HAVCR2 etc.

In certain embodiments, the bispecific molecule comprises a binding domain or a ligand for a TLR.

### Toll-Like Receptors (TLRs)

To date, 222 TLRs have been identified in invertebrates and 28 TLRs in vertebrates, with the highest number of TLRs in an individual found in the vertebrate fish of teleost which possesses 21 TLR molecules-TLR1-5, TLR7-9, TLR13-14, TLR18-23, and TLR25-28. In mammals, a total of 13 TLRs (TLR1-13) have been found-however, humans possess only 10 (TLR1-10) of them while mouse possesses all with TLR10 being nonfunctional (Sameer, Aga Syed, and Saniya Nissar. “Toll-Like Receptors (TLRs): Structure, Functions, Signaling, and Role of Their Polymorphisms in Colorectal Cancer Susceptibility.” BioMed research international vol. 2021 1157023. 12 Sep. 2021, doi: 10.1155/2021/1157023).

TLRs are homologous to the Toll receptor which was first identified in Drosophila melanogaster where it forms a complex with human nerve growth factor-like cystine knot protein Spätzle—the Toll-Spätzle complex-which is critical to both embryonic development and the generation of immune responses against fungi. Although structurally related to Drosophila TLR, molecular phylogenetic analyses have clarified that vertebrate TLR are highly conserved across various species and can be subclassified into six major families based on general class of pathogen-associated molecular patterns (PAMPs) recognized: TLR1 family which includes TLR1, TLR2, TLR6 and TLR10 (lipopeptide); TLR3 family [double stranded RNA (dsRNA)]; TLR4 family (lipopolysaccharide); TLR5 family (flagellin); TLR7-9 families [TLR7/8-single stranded RNA (ssRNA) and TLR9-double stranded DNA (dsDNA) or heme motifs]. The sixth remaining family-which includes TLR11-13 and TLR21-23 subfamilies—is represented in humans only as a pseudogene. Following synthesis within endoplasmic reticulum, traffic to Golgi, and proper folding, TLRs are either recruited to the cell surface (cell surface TLR) or intracellularly to endosomes (endosomal TLR)—a distribution that reflects the likelihood of exposure to the particular viral and/or bacterial nucleic acids.

TLRs are further subdivided into two groups, depending upon the number of cysteine clusters present within the extracellular LRR motif as follows: vertebrate type (V-Type) and protostome type (P-Type). V-Type TLRs possess a single cluster of cysteine or CF motif on their CTDs (LRRCT) and hence also referred to as Single Cysteine Cluster TLRs (sccTLRs), and P-Type TLRs have multiple (two or more) cysteine clusters or CF motifs either on their LRRCT or on the N-terminal (LRRNT) domain.

Cell surface TLRs include TLR1, TLR2, TLR4, TLR5, TLR6, whereas intracellular TLRs are localized in the endosome and include TLR3, TLR7, TLR8, TLR9, TLR10, TLR11, TLR12, and TLR13—although TLR11, TLR12, and TLR13 are not expressed in human tissues. Of the endosomal TLRs, TLR3 recognizes dsRNA, TLR7 and TLR8 recognize ssRNA while TLR9 recognizes dsDNA. Expression patterns of the endosomal TLRs further determine likelihood of encountering and responding to various PAMPS: TLR3 is expressed ubiquitously; TLR7 is expressed in lung, placenta and spleen; TLR8 is preferentially expressed in peripheral immune cells and lung tissue. In humans, TLR9s are predominantly expressed by antigen presenting cells (APC) particularly B cells, T cells and DCs within immune-cell-rich tissues including spleen, lymph node, and bone marrow. In humans, TLR10 is primarily expressed within endosomes although its function remains puzzling. Compelling data suggest that TLR10 exerts both pro-inflammatory and anti-inflammatory effects-former observed in the setting of viral infection, while the latter is mediated through a B cell-intrinsic process through antibody-mediated crosslinking. Overall, the ligand specificity and function of TLR10 remain less well characterized.

TLR7, TLR8 and TLR9 are primarily located in intracellular vesicles within the endoplasmic reticulum (ER) and translocate to endosomes upon stimulation by ligands. The release of TLR9 from the ER is controlled by several mechanisms including tyrosine-based motifs in TLR9 cytoplasmic tail, and phosphorylation of TLR9. Key proteins required for TLR9 traffic from ER to endosomal compartments include glycoprotein 96 (gp96), UNC93B1, adapter protein 3 (AP-3), a protein associated with TLR4 (PRAT4A), and Slc15a4. The unique localization and trafficking requirements of the nucleic acid-sensing TLRs 7-9 serve as a regulatory mechanism to limit immune responses to host nucleic acids; and indeed, artificial localization of TLR9 to cell surface causes autoimmune manifestations. After leaving the ER, TLR9 and other nucleic acid sensing TLRs traffic to the endosomal compartment where they are proteolytically processed.

TLR9 Structure and Ligands. Structurally, TLRs are type I transmembrane glycoproteins comprising an extracellular N-terminal ligand recognition domain, a single transmembrane helix, and an intracellular C-terminal cytoplasmic signaling domain. TLR extracellular domains (ECD) comprise repeated leucine-rich repeat (LRR) modules that bind PAMPs depending on TLR subtype. Each LRR module is 20-43 amino acids long and comprises a variable part and a highly conserved “LxxLxLxxN” motif where “L” is leucine, isoleucine, valine or phenylalanine and “N” is asparagine, threonine, serine or cysteine. LRR modules form one or two horseshoe domains wherein the “LxxLxLxxN” motifs are located in inner concave surfaces, while the variable parts form the outer convex surface. TLR receptor structure is characterized by constituent LRR motifs, repeat numbers and is flanked by two cysteine clusters including 2-4 cysteine residues across each TLR subtype. The TLR intracellular domain (ICD) comprises ˜150 amino acids and shares sequence homology with the signaling domains of IL-1R super-family, and hence is termed Toll/interleukin-1 receptor (TIR). The tertiary structures of TLR1-6 have been determined and have previously been summarized in other reviews on the topic.

Although TLRs 7-9 bear functional similarly to TLR3 in that they are localized intracellularly to endosomes and recognize nucleic acid PAMPs, the structures of TLRs 7-9 are markedly different from TLR3. The ECDs of TLRs 7-9 comprise 25 LRR modules, are heavily glycosylated, contain large insertions in LRRs 2, 5 and 8, and contain stretches of ˜40 amino acid residues between LRRs 14 and 15. The insertions arise from the glycan-free ECD surface involved in dimerization and hence, likely give rise to structures involved in dimerization; while the ˜40 residue stretches show a high degree of species variability and hence, are unlikely involved in dimerization. The TLR9 (and TLR7 and 8) ECD contains a Z-loop or hinge region between LRR14 and LRR15 where proteolytic cleavage by a cysteine lysosomal protease occurs to form proteolytically cleaved TLR9 (amino acids 471-1032) that maintains the horseshoe shape of the protomer. Both full length and proteolytically cleaved TLR9 (amino acids 471-1032) are predominantly monomeric in the absence of unmethylated cytidine phosphate guanosine (CpG). However, following ligand binding, proteolytically cleaved TLR9 dimerizes forming a homodimer.

TLR3, 4, 7/8 and 9 agonists are of particular interest as immunotherapeutic agents to treat cancer. Included in the group are, without limitation: 852A: Synthetic imidazoquinoline mimicking viral ssRNA; VTX-2337: Small-molecule selective TLR8 agonist mimicking viral ssRNA; BCG: Bacillus of Calmette-Guerin, Mycobacterium bovis; CpG ODN: CpG oligodeoxynucleotide; Imiquimod: Synthetic imidazoquinoline mimicking viral ssRNA; LPS: Lipopolysaccharide; MPL: Monophosphoryl lipid A; Poly I:C: Polyriboinosinic-polyribocytidylic acid; PolyICLC: Poly I:C-poly-1-lysine; Resiquimod: Synthetic imidazoquinoline mimicking viral ssRNA.

Imiquimod is a synthetic imidazoquinoline that targets TLR7. A newer imidazoquinoline TLR7 agonist, 852A, administered parenterally as monotherapy has shown modest clinical efficacy with disease stabilization as a monotherapy. Resiquimod is an imidazoquinoline TLR7/8 agonist in humans.

CpGs are single strand oligodeoxynucleotides (ODNs), characterized by motifs containing cytosines and guanines. TLR9 preferentially detects unmethylated CpG oligodinucleotides (ODN) with a species-specific preference for hexamer CpG motifs (human 5′-GTCGTT-3 vs. murine 5′-GACGTT-3) that are less common in vertebrate DNA. There are three major classes of CpG ODNs based on different backbones and sequence motifs. Type A CpG are characterized by poly G sequence at 5′ end, 3′ end or both; internal palindromic sequence containing GC nucleotides; and a partial phosphorothioate (PS) modified backbone. Type A CpG preferentially activate plasmacytoid DCs (pDC) and NK cells and induce significant IFN-α production by pDCs. Type B CpG are generally 18-28 nucleotides in length; have a complete PS-modified backbone; and contain one or more 6mer CpG motifs (5′-PuPyCGPyPu-3′) with the most potent ODNs in this class containing three 6mer sequences. Type B CpG preferentially activate B cells and less so NK cells with no effect on DCs. Type C CpG have features of both classes A and B: a complete PS-modified backbone and an internal palindromic motif. Consequently, the effects of type C CpG comprise features of both classes: strong direct B cell stimulation, IFN-α production by pDC, APC activation and maturation, and indirect NK cell activation.

Besides species specific ODN sequence preference for TLR9 activation, other factors including number and position of CpG motifs, nucleotides adjacent to CG dinucleotide, and ODN secondary structure influence ODN potency for TLR9 activation in humans. Synthetic TLR9 agonists, which are being used in clinical trials, have structural differences, which makes them nuclease resistant and also increases their half-life. A comprehensive list of synthetic TLR9 agonists categorized by type, structure and status of clinical evaluation is reviewed by Karapetyan L, Luke J J, Davar D. Toll-Like Receptor 9 Agonists in Cancer. Onco Targets Ther. 2020; 13:10039-10060. Published 2020 Oct. 9. doi: 10.2147/OTT.S247050 (incorporated in its entirety herein). Table 1 lists some synthetic agonists (Karapetyan L. et al.)

TABLE I
Toll-Like Receptor 9 Agonists Recently or Currently in Development

| TLR9 | | | | |
|---|---|---|---|---|
| Agonist | Structure | CpG Type | ROA | Tumor Types Being Evaluated |
| [symbol] | SNA with two components: (1) densely packed shell of | Undisclosed | [symbol] | Melanoma [symbol] MCC [symbol] HNSCC [symbol] cSCC [symbol] |
| [symbol] | CpG ODN radially oriented around (2) a core | | | advanced solid tumors |
| [symbol] | nanoparticle, which may be solid or hollow which | | | |
| | facilitates increased cellular uptake | | | |
| CMP-001 | [symbol] CpG ODN which forms G-quadruplex that mimics | A | [symbol] | Melanoma [symbol] NSCLC [symbol] CRC |
| (Checkmate) | [symbol] DNA encapsulated within 30 nm VLP comprised | | [symbol] | |
| | of capsid proteins derived from [symbol] known | | | |
| | as [symbol] | | | |
| CpG-28 | 5 [symbol] -TAAACGTTATAACGTTATGACGTCAT-3 [symbol] sequence | [symbol] | Intracerebral | [symbol] |
| (University of | with 3 CpG motifs and a fully [symbol] backbone | | infusion | [symbol] |
| Paris) | | | | |
| no longer in | | | | |
| development) | | | | |
| [symbol] | Member of the dSLIM family exhibiting a double stem of 28 | Undisclosed | [symbol] | Preclinical development |
| ( [symbol] AG) | base pairs and two loops with 30 nucleotides [symbol] each | | [symbol] | |
| (no longer in | containing three CG motifs [symbol] that has a linear structure | | | |
| development) | | | | |
| IMO-2055 | [symbol] sequence with two CpG ODN [symbol] | [symbol] | [symbol] | Melanoma [symbol] NSCLC [symbol] CRC [symbol] |
| [symbol] | synthetic immunostimulatory motifs and two 5′ ends for | | [symbol] | HNSCC [symbol] and refractory solid |
| (no longer in | increased metabolic [symbol] | | | tumors |
| development) | | | | |
| [symbol] | [symbol] sequence with three CpG ODN | [symbol] | [symbol] | Melanoma [symbol] CRC |
| [symbol] | | | | |
| [symbol] | | | | |
| [symbol] | Member of the dSUM family exhibiting a double [symbol] of 28 | Undisclosed | [symbol] | Melanoma [symbol] CRC [symbol] RCC [symbol] advanced |
| [symbol] | base pairs and two loops with 30 nucleotides [symbol] each | | [symbol] | solid rumors |
| [symbol] | containing three CG motifs [symbol] that is dumbbell-shaped and | | | |
| (no longer in | [symbol] | | | |
| development) | | | | |
| [symbol] | Undisclosed [symbol] structure encapsulated within a calcium | Undisclosed | Undisclosed | Preclinical development |
| [symbol] | sulfate excipient (NanoZolid) that permits slow release | | | |
| | depot | | | |
| [symbol] | [symbol] backbone with one or more CpG | [symbol] | [symbol] | [symbol] |
| (Pfizer) | [symbol] | | [symbol] | CTCL [symbol] NHL |
| (no longer in | | | [symbol] | |
| development) | | | | |
| [symbol] | 30 nucleotide [symbol] sequence with multiple | C | [symbol] | Melanoma [symbol] lymphoma [symbol] HNSCC [symbol] |
| [symbol] | CpG motifs and internal [symbol] sequences | | [symbol] | prostate cancer [symbol] breast cancer [symbol] |
| (no longer in | | | | refractory solid tumors |
| development) | | | | |
| 5-540956 | Undisclosed | Undisclosed | Undisclosed | Preclinical development |
| (Shionogi) | | | | |

Abbreviations: [symbol] [symbol]
indicates data missing or illegible when filed

In certain embodiments, a TLR9 agonist comprises a substituted CpG oligonucleotide. Certain synthetic modifications of C or G substituted within a CpG motif are recognized by TLR9. These substituted motifs are referred to as synthetic immune stimulatory motifs CpR and YpG. Other examples include TLR9 agonists substituted with synthetic pyrimidines, such as 5-OH-dC, 5-propyne-dC, furano-dT, 1-(2′-deoxy-β-d-ribofuranosyl)-2-oxo-7-deaza-8-methylpurine, dF, 4-thio-dU, N³-Me-dC, N⁴-Et-dC, Φ-iso-dC, and arabinoC, in place of C in the CpG. TLR9 agonists substituted with synthetic purines, such as 7-deaza-dG, 7-deaza-8-aza-dG, 9-deaza-dG, N¹-Me-dG, N²-Me-dG, 6-Thio-dG, dI, 8-OMe-dG, 8-O-allyl-dG, and arabinoG bases, in place of G in the CpG dinucleotide (D. Yu et al., Agonists of Toll-like Receptor 9 Containing Synthetic Dinucleotide Motifs. Journal of Medicinal Chemistry, 2007, 50 (25), 6411-6418. DOI: 10.1021/jm0708811.

### Antibody Production

In some embodiments, polypeptides described herein (e.g., antibodies and its binding fragments) are produced using any method known in the art to be useful for the synthesis of polypeptides (e.g., antibodies) by chemical synthesis or by recombinant expression, and are preferably produced by recombinant expression techniques.

In some embodiments, an antibody or its binding fragment thereof is expressed recombinantly, and the nucleic acid encoding the antibody or its binding fragment is assembled from chemically synthesized oligonucleotides (e.g., as described in Kutmeier et al., 1994, BioTechniques 17:242), which involves the synthesis of overlapping oligonucleotides containing portions of the sequence encoding the antibody, annealing and ligation of those oligonucleotides, and then amplification of the ligated oligonucleotides by PCR.

Alternatively, a nucleic acid molecule encoding an antibody is optionally generated from a suitable source (e.g., an antibody cDNA library, or cDNA library generated from any tissue or cells expressing the immunoglobulin) by PCR amplification using synthetic primers hybridizable to 3′ and 5′ ends of the sequence or by cloning using an oligonucleotide probe specific for the particular gene sequence.

In some embodiments, an antibody or its binding is optionally generated by immunizing an animal, such as a rabbit, to generate polyclonal antibodies or, more preferably, by generating monoclonal antibodies, e.g., as described by Kohler and Milstein (1975, Nature 256:495-497) or, as described by Kozbor et al. Alternatively, a clone encoding at least the Fab portion of the antibody is optionally obtained by screening Fab expression libraries (e.g., as described in Huse et al., 1989, Science 246:1275-1281) for clones of Fab fragments that bind the specific antigen or by screening antibody libraries (See, e.g., Clackson et al., 1991, Nature 352:624; Hane et al., 1997 Proc. Natl. Acad. Sci. USA 94:4937).

Polyclonal antibodies can be raised by a standard protocol by injecting a production animal with an antigenic composition. See, e.g., Harlow and Lane, Antibodies: A Laboratory Manual, Cold Spring Harbor Laboratory, 1988. When utilizing an entire protein, or a larger section of the protein, antibodies may be raised by immunizing the production animal with the protein and a suitable adjuvant (e.g., Freund's, Freund's complete, oil-in-water emulsions, etc.) When a smaller peptide is utilized, it is advantageous to conjugate the peptide with a larger molecule to make an immunostimulatory conjugate. Commonly utilized conjugate proteins that are commercially available for such use include bovine serum albumin (BSA) and keyhole limpet hemocyanin (KLH). In order to raise antibodies to particular epitopes, peptides derived from the full sequence may be utilized. Alternatively, in order to generate antibodies to relatively short peptide portions of the protein target, a superior immune response may be elicited if the polypeptide is joined to a carrier protein, such as ovalbumin, BSA or KLH.

Alternatively, for monoclonal antibodies, hybridomas may be formed by isolating the stimulated immune cells, such as those from the spleen of the inoculated animal. These cells are then fused to immortalized cells, such as myeloma cells or transformed cells, which are capable of replicating indefinitely in cell culture, thereby producing an immortal, immunoglobulin-secreting cell line. In addition, the antibodies or antigen binding fragments may be produced by genetic engineering. Humanized, chimeric, or xenogeneic human antibodies, which produce less of an immune response when administered to humans, are preferred for use in the present disclosure.

Antibodies that have a reduced propensity to induce a violent or detrimental immune response in humans (such as anaphylactic shock), and which also exhibit a reduced propensity for priming an immune response which would prevent repeated dosage with the antibody therapeutic or imaging agent are preferred for use in the disclosure. These antibodies are preferred for all administrative routes. Thus, humanized, chimeric, or xenogenic human antibodies, which produce less of an immune response when administered to humans, are preferred for use in the present disclosure.

Chimeric antibodies may be made by recombinant means by combining the murine variable light and heavy chain regions obtained from a murine (or other animal-derived) hybridoma clone, with the human constant light and heavy chain regions, in order to produce an antibody with predominantly human domains. The production of such chimeric antibodies is well known in the art and may be achieved by standard means (as described, e.g., in U.S. Pat. No. 5,624,659, incorporated fully herein by reference). Humanized antibodies are engineered to contain even more human-like immunoglobulin domains and incorporate only the complementarity determining regions of the animal-derived antibody. This is accomplished by carefully examining the sequence of the hyper-variable loops of the variable regions of the monoclonal antibody and fitting them to the structure of the human antibody chains. Although facially complex, the process is straightforward in practice. See, e.g., U.S. Pat. No. 6,187,287, incorporated fully herein by reference. Alternatively, single chain antibodies (Fv, as described below) can be produced from phage libraries containing human variable regions. See U.S. Pat. No. 6,174,708, incorporated fully herein by reference.

In addition to entire immunoglobulins (or their recombinant counterparts), immunoglobulin fragments comprising the epitope binding site (e.g., Fab′, F(ab′)₂, or other fragments) are useful as antibody moieties in the present disclosure. Such antibody fragments may be generated from whole immunoglobulins by ficin, pepsin, papain, or other protease cleavage. “Fragment” or minimal immunoglobulins may be designed utilizing recombinant immunoglobulin techniques. For instance, “Fv” immunoglobulins for use in the present disclosure may be produced by linking a variable light chain region to a variable heavy chain region via a peptide linker (e.g., poly-glycine or another sequence which does not form an alpha helix or beta sheet motif).

Fv fragments are heterodimers of the variable heavy chain domain (V_{H}) and the variable light chain domain (V_{L}). The heterodimers of heavy and light chain domains that occur in whole IgG, for example, are connected by a disulfide bond. Recombinant Fvs in which V_{H} and V_{L} are connected by a peptide linker are typically stable, see, for example, Huston et al., Proc. Natl. Acad, Sci. USA 85:5879-5883 (1988) and Bird et al., Science 242:423-426 (1988), both fully incorporated herein, by reference. These are single chain Fvs which have been found to retain specificity and affinity and have been shown to be useful for imaging tumors and to make recombinant immunotoxins for tumor therapy. Any of these minimal antibodies may be utilized in the present disclosure, and those which are humanized to avoid HAMA reactions can be used in embodiments of the disclosure.

In addition, derivatized immunoglobulins with added chemical linkers, detectable moieties, e.g., fluorescent dyes, enzymes, radioisotopes, substrates, chemiluminescent moieties, or specific binding moieties, e.g. streptavidin, avidin, biotin, etc. may be utilized in the methods and compositions of the present disclosure. For convenience, the term “antibody” or “antibody moiety” will be used throughout to generally refer to molecules which specifically bind to an epitope of the targeted protein, although the term will encompass all immunoglobulins, derivatives, fragments, recombinant or engineered immunoglobulins, and modified immunoglobulins, as described above.

Candidate binding agents can be tested for activity by any suitable standard means. As a first screen, the antibodies may be tested for binding against the target antigen utilized to produce them. As a second screen, candidate agents may be tested for binding to an appropriate cell, e.g. cancer cell, hematopoietic cell, etc. For these screens, the candidate antibody may be labeled for detection (e.g., with fluorescein or another fluorescent moiety, or with an enzyme such as horseradish peroxidase). After selective binding to the target is established, the candidate agent may be tested for appropriate activity (i.e., the ability to decrease tumor cell growth and/or to aid in visualizing tumor cells) in an in vivo model.

In some embodiments, an expression vector comprising the nucleotide sequence of an antibody, or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In specific embodiments, the expression of the antibody is regulated by a constitutive, an inducible or a tissue, specific promoter.

In some embodiments, a variety of host-expression vector systems is utilized to express an antibody, or its binding fragment described herein. Such host expression systems represent vehicles by which the coding sequences of the antibody is produced and subsequently purified, but also represent cells that are, when transformed or transfected with the appropriate nucleotide coding sequences, express an antibody or its binding fragment in situ. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces, Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression is preferred. In some embodiments, cell lines that stably express an antibody are optionally engineered. Rather than using expression vectors that contain viral origins of replication, host cells are transformed with DNA controlled by appropriate expression control elements (e.g., promoter, enhancer, sequences, transcription terminators, polyadenylation sites, etc.), and a selectable marker. Following the introduction of the foreign DNA, engineered cells are then allowed to grow for 1-2 days in an enriched media, and then are switched to a selective media. The selectable marker in the recombinant plasmid confers resistance to the selection and allows cells to stably integrate the plasmid into their chromosomes and grow to form foci that in turn are cloned and expanded into cell lines. This method can advantageously be used to engineer cell lines which express the antibody or its binding fragments.

In some embodiments, a number of selection systems are used, including but not limited to the herpes simplex virus thymidine kinase, hypoxanthine-guanine phosphoribosyltransferase, and adenine phosphoribosyltransferase genes are employed in tk-, hgprt- or aprt-cells, respectively. Also, antimetabolite resistance are used as the basis of selection for the following genes: dhfr, which confers resistance to methotrexate; gpt, which confers resistance to mycophenolic acid; neo, which confers resistance to the aminoglycoside G-418 and hygro, which confers resistance to hygromycin. Methods commonly known in the art of recombinant DNA technology which can be used are described in Ausubel et al. (eds., 1993, Current Protocols in Molecular Biology, John Wiley & Sons, NY; Kriegler, 1990, Gene Transfer and Expression, A Laboratory Manual, Stockton Press, NY; and in Chapters 12 and 13, Dracopoli et al. (eds), 1994, Current Protocols in Human Genetics, John Wiley & Sons, NY.; Colberre-Garapin et al., 1981, J. Mol. Biol. 150:1).

In some embodiments, the expression levels of an antibody are increased by vector amplification. When a marker in the vector system expressing an antibody is amplifiable, an increase in the level of inhibitor present in culture of host cell will increase the number of copies of the marker gene. Since the amplified region is associated with the nucleotide sequence of the antibody, production of the antibody will also increase.

In some embodiments, any method known in the art for purification of an antibody is used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

In some embodiments, vectors include any suitable vectors derived from either a eukaryotic or prokaryotic sources. In some embodiments, vectors are obtained from bacteria (e.g., E. coli), insects, yeast (e.g., Pichia pastoris), algae, or mammalian sources. Exemplary bacterial vectors include pACYC177, pASK75, pBAD vector series, pBADM vector series, pET vector series, pE™ vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some embodiments, yeast vectors include Gateway® pDEST™ 14 vector, Gateway® pDEST™ 15 vector, Gateway® pDEST™. 17 vector, Gateway® pDEST™ 24 vector, Gateway® pYES-DEST52 vector, pBAD-DEST49 Gateway® destination vector, pAO815 Pichia vector, pFLDI Pichia pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include p3xFLAG-CMV 8, pFLAG-Myc-CMV 19, pFLAG-Myc-CMV 23, pFLAG-CMV 2, pFLAG-CMV 6a, b, c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some embodiments, a cell-free system is a mixture of cytoplasmic and/or nuclear components from a cell and is used for in vitro nucleic acid synthesis. In some embodiments, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

In some embodiments, a host cell includes any suitable cell such as a naturally derived cell or a genetically modified cell. In some embodiments, a host cell is a production host cell. In some embodiments, a host cell is a eukaryotic cell. In other embodiments, a host cell is a prokaryotic cell. In some embodiments, a eukaryotic cell includes fungi (e.g., yeast cells), animal cell or plant cell. In some embodiments, a prokaryotic cell is a bacterial cell. Examples of bacterial cell include gram-positive bacteria or gram-negative bacteria. Sometimes the gram-negative bacteria are anaerobic, rod-shaped, or both.

In some embodiments, gram-positive bacteria include Actinobacteria, Firmicutes or Tenericutes. In some embodiments, gram-negative bacteria include Aquificae, Deinococcus-Thermus, Fibrobacteres-Chlorobi Bacteroidetes (FCB group), Fusobacteria, Gemmatimonadetes, Nitrospirae, Planctomycetes-Verrucomicrobia Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™ DH10B™, TOP10, DH5a, DH10Bac™, OmniMax™, MegaX™, DH12S™, INV110, TOP1OF′, INVαF, OP10/P3, ccdB Survival, PIR1, PIR2, Stb12™, Stb13™, or Stb14™

In some embodiments, animal cells include a cell from a vertebrate or from an invertebrate. In some embodiments, an animal cell includes a cell from a marine invertebrate, fish, insects, amphibian, reptile, or mammal. In some embodiments, a fungus cell includes a yeast cell, such as brewer's yeast, baker's yeast, or wine yeast.

Fungi include ascomycetes such as yeast, mold, filamentous fungi, basidiomycetes, or zygomycetes. In some embodiments, yeast includes Ascomycota or Basidiomycota. In some embodiments, Ascomycota includes Saccharomycotina (true yeasts, e.g. Saccharomyces cerevisiae (baker's yeast)) or Taphrinomycotina (e.g. Schizosaccharomyces (fission yeasts)). In some embodiments, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

In some embodiments, additional animal cells include cells obtained from a mollusk, arthropod, annelid or sponge. In some embodiments, an additional animal cell is a mammalian cell, e.g., from a primate, ape, equine, bovine, porcine, canine, feline or rodent. In some embodiments, a rodent includes mouse, rat, hamster, gerbil, hamster, chinchilla, fancy rat, or guinea pig.

Exemplary mammalian host cells include, but are not limited to, 293A cell line, 293 FT cell line, 293F cells, 293 H cells, CHO DG44 cells, CHO-S cells, CHO-K1 cells, Expi293F™ cells, Flp-In™, T-Rex™, 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-Rex™ Jurkat cell line, Per.C6 cells, T-Rex™-293 cell line, T-Rex™-CHO cell line, and T-Rex™-HeLa cell line.

In some embodiments, a mammalian host cell is a stable cell line, or a cell line that has incorporated a genetic material of interest into its own genome and has the capability to express the product of the genetic material after many generations of cell division. In some embodiments, a mammalian host cell is a transient cell line, or a cell line that has not incorporated a genetic material of interest into its own genome and does not have the capability to express the product of the genetic material after many generations of cell division. Exemplary insect host cell include, but are not limited to, Drosophila S2 cells, Sf9 cells, Sf21 cells, High Five™ cells, and expresSF+® cells.

In some embodiments, plant cells include a cell from algae. Exemplary insect cell lines include, but are not limited to, strains from Chlamydomonas reinhardtii 137c, or Synechococcus elongatus PPC 7942.

### Pharmaceutical Compositions and Formulations

For therapeutic use, the chimeric molecules embodied herein are combined with a pharmaceutically acceptable carrier. As used herein, “pharmaceutically acceptable carrier” means buffers, carriers, and excipients suitable for use in contact with the tissues of human beings and animals without excessive toxicity, irritation, allergic response, or other problem or complication, commensurate with a reasonable benefit/risk ratio. The carrier(s) should be “acceptable” in the sense of being compatible with the other ingredients of the formulations and not deleterious to the recipient. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

Accordingly, compositions of the present disclosure can comprise at least one of any suitable excipients, such as, but not limited to, diluent, binder, stabilizer, buffers, salts, lipophilic solvents, preservative, adjuvant or the like. Pharmaceutically acceptable excipients are preferred. Non-limiting examples of, and methods of preparing such sterile solutions are well known in the art, such as, but not limited to, those described in Gennaro, Ed., Remington's Pharmaceutical Sciences, 18th Edition, Mack Publishing Co. (Easton, Pa.) 1990. Pharmaceutically acceptable carriers can be routinely selected that are suitable for the mode of administration, solubility and/or stability of the antibody molecule, fragment or variant composition as well known in the art or as described herein.

Pharmaceutical excipients and additives useful in the present composition include but are not limited to proteins, peptides, amino acids, lipids, and carbohydrates (e.g., sugars, including monosaccharides, di-, tri-, tetra-, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Representative amino acid/antibody molecule components, which can also function in a buffering capacity, include alanine, glycine, arginine, betaine, histidine, glutamic acid, aspartic acid, cysteine, lysine, leucine, isoleucine, valine, methionine, phenylalanine, aspartame, and the like.

Carbohydrate excipients suitable for use in the disclosure include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol), myoinositol and the like. Preferred carbohydrate excipients for use in the present disclosure are mannitol, trehalose, and raffinose.

Antibody molecule compositions can also include a buffer or a pH adjusting agent; typically, the buffer is a salt prepared from an organic acid or base. Representative buffers include organic acid salts such as salts of citric acid, acetic acid, ascorbic acid, gluconic acid, carbonic acid, tartaric acid, succinic acid, or phthalic acid; Tris, tromethamine hydrochloride, or phosphate buffers.

Additionally, antibody molecule compositions of the disclosure can include polymeric excipients/additives such as polyvinylpyrrolidones, ficolls (a polymeric sugar), dextrates (e.g., cyclodextrins, such as 2-hydroxypropyl-.beta.-cyclodextrin), polyethylene glycols, flavoring agents, antimicrobial agents, sweeteners, antioxidants, antistatic agents, surfactants (e.g., polysorbates such as “TWEEN 20” and “TWEEN 80”), lipids (e.g., phospholipids, fatty acids), steroids (e.g., cholesterol), and chelating agents (e.g., EDTA).

These and additional known pharmaceutical excipients and/or additives suitable for use in the antibody molecule compositions according to the disclosure are known in the art, e.g., as listed in “Remington: The Science & Practice of Pharmacy,” 19th ed., Williams & Williams, (1995), and in the “Physician's Desk Reference,” 52nd ed., Medical Economics, Montvale, N.J. (1998). Preferred carrier or excipient materials are carbohydrates (e.g., saccharides and alditols) and buffers (e.g., citrate) or polymeric agents.

The present disclosure provides for stable compositions, comprising at least one anti-CD47 antibody molecule in a pharmaceutically acceptable formulation. Preserved formulations contain at least one known preservative or optionally selected from at least one phenol, m-cresol, p-cresol, o-cresol, chlorocresol, benzyl alcohol, phenylmercuric nitrite, phenoxyethanol, formaldehyde, chlorobutanol, magnesium chloride (e.g., hexahydrate), alkylparaben (methyl, ethyl, propyl, butyl and the like), benzalkonium chloride, benzethonium chloride, sodium dehydroacetate and thimerosal, or mixtures thereof in an aqueous diluent. Any suitable concentration or mixture can be used as known in the art, such as 0.001-5%, or any range or value therein, such as, but not limited to 0.001, 0.003, 0.005, 0.009, 0.01, 0.02, 0.03, 0.05, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.3, 4.5, 4.6, 4.7, 4.8, 4.9, or any range or value therein. Non-limiting examples include, no preservative, 0.1-2% m-cresol (e.g., 0.2, 0.3, 0.4, 0.5, 0.9, or 1.0%), 0.1-3% benzyl alcohol (e.g., 0.5, 0.9, 1.1., 1.5, 1.9, 2.0, or 2.5%), 0.001-0.5% thimerosal (e.g., 0.005 or 0.01%), 0.001-2.0% phenol (e.g., 0.05, 0.25, 0.28, 0.5, 0.9, or 1.0%), 0.0005-1.0% alkylparaben(s) (e.g., 0.00075, 0.0009, 0.001, 0.002, 0.005, 0.0075, 0.009, 0.01, 0.02, 0.05, 0.075, 0.09, 0.1, 0.2, 0.3, 0.5, 0.75, 0.9, or 1.0%), and the like.

Pharmaceutical compositions containing chimeric molecules as disclosed herein can be presented in a dosage unit form and can be prepared by any suitable method. A pharmaceutical composition should be formulated to be compatible with its intended route of administration. Examples of routes of administration are intravenous (IV), intradermal, inhalation, transdermal, topical, transmucosal, and rectal administration. A preferred route of administration for monoclonal antibodies is IV infusion. Useful formulations can be prepared by methods known in the pharmaceutical art. For example, see Remington's Pharmaceutical Sciences (1990) supra. Formulation components suitable for parenteral administration include a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

For intravenous administration, suitable carriers include physiological saline, bacteriostatic water, Cremophor EL™ (BASF, Parsippany, N.J.) or phosphate buffered saline (PBS). The carrier should be stable under the conditions of manufacture and storage and should be preserved against microorganisms. The carrier can be a solvent or dispersion medium containing, for example, water, ethanol, polyol (for example, glycerol, propylene glycol, and liquid polyethylene glycol), and suitable mixtures thereof.

Pharmaceutical formulations are preferably sterile. Sterilization can be accomplished by any suitable method, e.g., filtration through sterile filtration membranes. Where the composition is lyophilized, filter sterilization can be conducted prior to or following lyophilization and reconstitution.

The compositions of this disclosure may be in a variety of forms. These include, for example, liquid, semi-solid and solid dosage forms, such as liquid solutions (e.g., injectable and infusible solutions), dispersions or suspensions, and liposomes. The preferred form depends on the intended mode of administration and therapeutic application. Typical preferred compositions are in the form of injectable or infusible solutions. The preferred mode of administration is parenteral (e.g., intravenous, subcutaneous, intraocular, intraperitoneal, intramuscular). In a preferred embodiment, the preparation is administered by intravenous infusion or injection. In another preferred embodiment, the preparation is administered by intramuscular or subcutaneous injection.

The phrases “parenteral administration” and “administered parenterally” as used herein means modes of administration other than enteral and topical administration, usually by injection, and includes, without limitation, intravenous, intramuscular, subcutaneous, intraarterial, intrathecal, intracapsular, intraorbital, intravitreous, intracardiac, intradermal, intraperitoneal, transtracheal, inhaled, subcutaneous, subcuticular, intraarticular, subcapsular, subarachnoid, intraspinal, epidural and intrasternal injection and infusion.

The present disclosure provides a kit, comprising packaging material and at least one vial comprising a solution of at least one chimeric molecule with the prescribed buffers and/or preservatives, optionally in an aqueous diluent. The aqueous diluent optionally further comprises a pharmaceutically acceptable preservative. Preservatives include those selected from phenol, m-cresol, p-cresol, o-cresol, chlorocresol, benzyl alcohol, alkylparaben (methyl, ethyl, propyl, butyl and the like), benzalkonium chloride, benzethonium chloride, sodium dehydroacetate and thimerosal, or mixtures thereof. The concentration of preservative used in the formulation is a concentration sufficient to yield an anti-microbial effect. Such concentrations are dependent on the preservative selected and are readily determined by the skilled artisan.

Other excipients, e.g. isotonicity agents, buffers, antioxidants, preservative enhancers, can be optionally and preferably added to the diluent. An isotonicity agent, such as glycerin, is commonly used at known concentrations. A physiologically tolerated buffer is preferably added to provide improved pH control. The formulations can cover a wide range of pHs, such as from about pH 4.0 to about pH 10.0, from about pH 5.0 to about pH 9.0, or about pH 6.0 to about pH 8.0

Other additives, such as a pharmaceutically acceptable solubilizers like TWEEN 20 (polyoxyethylene (20) sorbitan monolaurate), TWEEN 40 (polyoxyethylene (20) sorbitan monopalmitate), TWEEN 80 (polyoxyethylene (20) sorbitan monooleate), Pluronic F68 (polyoxyethylene polyoxypropylene block copolymers), and PEG (polyethylene glycol) or non-ionic surfactants such as polysorbate 20 or 80 or poloxamer 184 or 188, Pluronic® polyls, other block co-polymers, and chelators such as EDTA and EGTA can optionally be added to the formulations or compositions to reduce aggregation. These additives are particularly useful if a pump or plastic container is used to administer the formulation. The presence of pharmaceutically acceptable surfactant mitigates the propensity for the protein to aggregate.

Various delivery systems can be used to administer chimeric molecules to a subject. In certain exemplary embodiments, administration of chimeric moelcule is by intravenous infusion. In some embodiments, administration is by a two hour intravenous infusion.

Any of the formulations described above can be stored in a liquid or frozen form and can be optionally subjected to a preservation process. In some embodiments, the formulations described above are lyophilized, i.e., they are subjected to lyophilization. In some embodiments, the formulations described above are subjected to a preservation process, for example, lyophilization, and are subsequently reconstituted with a suitable liquid, for example, water. By lyophilized, it is meant that the composition has been freeze-dried under a vacuum. Lyophilization typically is accomplished by freezing a particular formulation such that the solutes are separated from the solvent(s). The solvent is then removed by sublimation (i.e., primary drying) and next by desorption (i.e., secondary drying).

The formulations of the present disclosure can be used with the methods described herein or with other methods for treating disease. The formulations may be further diluted before administration to a subject. In some embodiments, the formulations will be diluted with saline and held in IV bags or syringes before administration to a subject. Accordingly, in some embodiments, the methods for treating a CD47-expressing cancer in a subject will comprise administering to a subject in need thereof a weekly dose of a pharmaceutical composition comprising an anti-CD47 antibody or masked antibody.

It will be readily apparent to those skilled in the art that other suitable modifications and adaptations of the methods described herein may be made using suitable equivalents without departing from the scope of the embodiments disclosed herein. Having now described certain embodiments in detail, the same will be more clearly understood by reference to the following examples, which are included for purposes of illustration only and are not intended to be limiting. All patents, patent applications and references described herein are incorporated by reference in their entireties for all purposes.

### Examples

### Example 1: αCD47-CpG Conjugate

Cluster of differentiation 47 (CD47) is a myeloid checkpoint upregulated in numerous hematologic and solid malignancies to evade host immune response. Specifically, by interacting with signal regulatory protein-α (SIRPα), CD47 conveys ‘don't-eat-me’ signal to phagocytic cells to evade phagocytosis (4, 5). Tumor cells in multiple NHL subsets overexpress CD47, which serves as an independent predictor for adverse clinical outcomes (4, 6, 7). Thus, CD47-SIRPα has emerged as an attractive therapeutic target, and several inhibitory strategies, including αCD47, are actively investigated (6-8). Early in vitro and preclinical studies have collectively demonstrated that CD47 blockade induces phagocytosis of various tumor cells, including NHL cells (6, 8). More recently, αCD47 treatment was shown to promote cross-priming of CD8⁺ effector T cells, underscoring its potential role in adaptive immunity (7, 9). Nevertheless, clinical studies exploring CD47 blockade as a monotherapy to date revealed limited therapeutic efficacy across multiple cancer types (8). In contrast, combination of CD47 blockade and rituximab was recently shown in independent clinical trials (NCT2953509, NCT02367196, NCT03013218) to provide meaningful therapeutic activity without incurring clinically significant safety issues among NHL patients (3). However, unlike the more drastic synergistic effect observed in a preclinical study (4), clinical benefits were found to be moderate with the objective response rates spanning 13%-50% (3).

Of note, the modest therapeutic efficacy was observed in these studies despite near 100% CD47-receptor occupancy and the complementary ‘eat-me’ signaling provided by rituximab (3). The findings indicate that the direct tumor cell killing by macrophages is likely insufficient in eradicating non-Hodgkin's lymphoma (NHL). NHL is a highly prevalent and aggressive form of lymphoid cell malignancy. Indeed, tumor-specific T-cell reaction is an important component of CD47 blockade-induced tumor control, including the production of immune memory to inhibit tumor recurrence (7, a10-12). On the other hand, accumulated evidence underscores an essential role of toll-like receptor (TLR) 9 agonists on promoting the activation of antigen presenting cells and thus adaptive immune response (13). Among TLR9 agonists, CpG has been most widely explored in clinical studies and human safety has been established as monotherapy (13). A primary challenge to successfully implementing the CpG-based drugs is their short half-life (14). Without wishing to be bound by theory, it was hypothesized that by piggybacking on long circulating monoclonal antibody (mAb) drugs would address the issue.

To this end, novel immune stimulating antibody conjugate (ISAC) was developed and is composed of monoclonal antibody (mAb) against CD47 (αCD47) and unmethylated cytidine phosphate guanosine (CpG), termed herein αCD47-CpG, for immunotherapy of hematologic and solid tumors. Clinical development of αCD47-CpG, will provide a novel anti-cancer immunotherapy modality against NHL and other cancers, that comprehensively modulates both innate and adaptive immune responses.

### Results

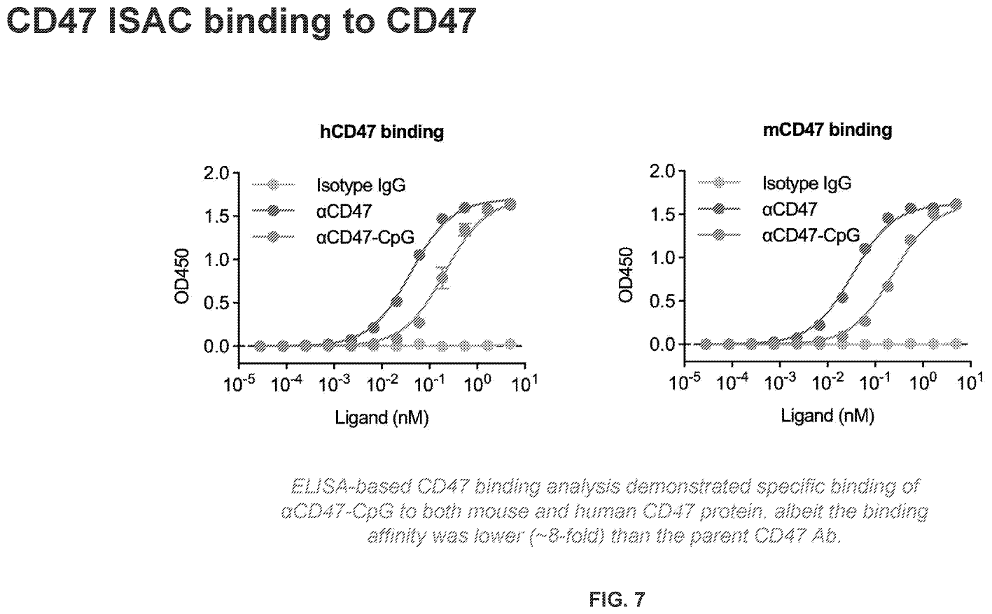

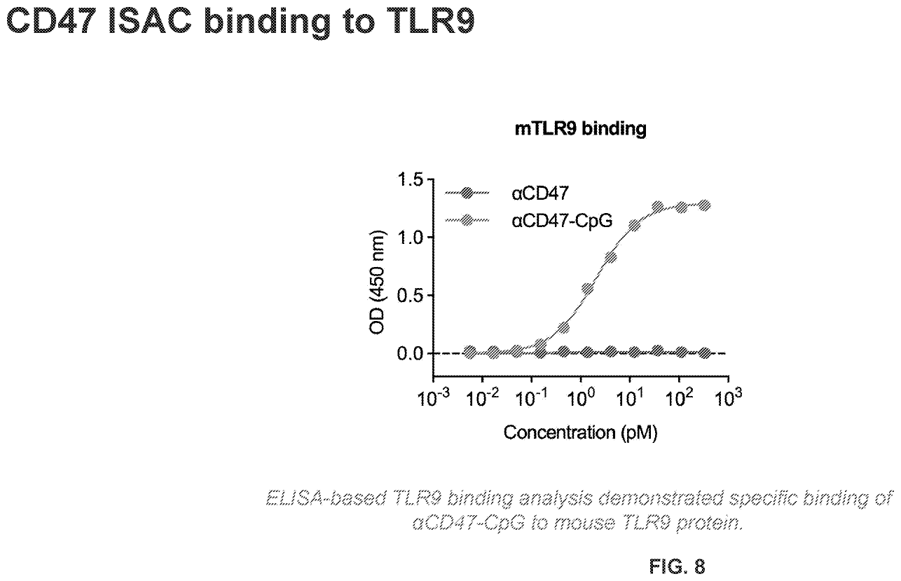

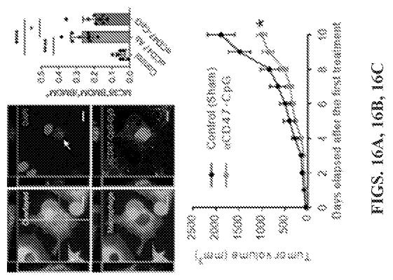

The engineered αCD47-CpG chimeric molecule demonstrated that the conjugate retains the activities of both αCD47 and CpG in vitro. Briefly, the CpG molecules were conjugated to mouse αCD47 at a drug-antibody ratio (DAR) of 8 and the conjugation was confirmed by SDS-PAGE analysis and size exclusion chromatography (SEC). It was first demonstrated that the &CD47-CpG binds to human/mouse CD47 and mouse TLR9 in a dose-dependent manner with nano-and picomolar binding affinity, respectively (FIGS. 7 and 8). It was then confirmed that αCD47-CpG induced NF-κB activity in mouse macrophages in monoculture and in co-culture with A20 mouse lymphoma cells to a degree on par with CpG, indicating that the biological activity of CpG in the conjugate was fully retained. Likewise, αCD47-CpG promoted phagocytosis of cancer cells by macrophages although the level was slightly lower compared to αCD47 (FIGS. 16A, 16B), reflecting the modest reduction of the binding affinity by the conjugation (FIGS. 7A, 7B). Importantly, αCD47-CpG enhanced the release of pro-inflammatory cytokines bridging the innate and adaptive immune responses in a macrophage-cancer cell co-culture over individual components and their physical mixture. A pilot study was conducted in vivo using a flank tumor model in which was found that αCD47-CpG significantly delayed the tumor growth (FIG. 16C) while αCD47 alone failed to do so. These data demonstrate that the αCD47-CpG can serve as a potent immunomodulatory therapy of NHL and other cancers.

### Example 2: Optimization of the αCD47-CpG Conjugate

We will engineer 9 conjugate candidates at varying DAR, specifically 2, 4, 6, and 8, using the thiol-maleimide chemistry, and confirm the conjugation via SDS-PAGE analysis and SEC. We will next determine the binding affinity of the conjugate candidates to CD47 and TLR9 immobilized on solid supports using ELISA assay. We will also investigate the ability of the conjugate candidates to promote innate and adaptive immune responses in vitro, by assessing NF-κB activity, phagocytosis index, pro-inflammatory cytokine production, antigen cross-presentation, and T-cell priming. As an in vitro safety measure, we will test whether the conjugates induce erythrocyte agglutination.

### Example 3: Investigation of In Vivo Immunomodulatory Effects, Anti-Cancer Efficacy and Safety of the @CD47-CpG

It is critical that the anti-cancer biological activities of the αCD47-CpG conjugate demonstrated in vitro translate to promising therapeutic outcomes in vivo. In addition, potential systemic adverse effects previously observed in early clinical trials exploring the anti-CD47 strategies should be carefully monitored.

We will first conduct a dose escalation study to determine the maximum tolerated dose (MTD) using the lead αCD47-CpG candidate selected in Aim 1. Specifically, we will treat healthy inbred mice (i.e., BALB/c mice) with three escalating doses of the lead candidate via systemic administration (i.e., tail vein injection), followed by complete blood count analysis to determine potential adverse effects, specifically anemia and thrombocytopenia. We will employ the clinically validated low-dose priming strategy that selectively eliminates aging red blood cells while sparing younger cells lacking prophagocytic signals if the tested doses exhibit significant adverse effects.

We will then conduct in vivo immunomodulatory and anti-cancer efficacy studies using two complementary mouse models of B-cell lymphoma, the most common type of NHL in the United States, based on A20 lymphoma cells at the MTD with an optional priming. The first model will be established by subcutaneous inoculation of A20 or ovalbumin (OVA)-expressing A20 cells that allows monitoring tumor growth and immunological tumor microenvironment. When the tumor volume reaches a certain size (i.e., 50 mm³), the lead αCD47-CpG conjugate will be given via systemic administration at the MTD every third day up to four doses. For comparison, animals in different groups will received either PBS, αCD47 or CpG following the identical dosing schedule. Throughout the course of the treatments and afterwards, the tumor volume will be measured and subsequently tumor tissues and spleen will be harvested for immune cell and/or cytokine profiling. Important cell populations to be monitored are tumor-associated macrophages and CD4⁺/CD8⁺ T cells, including OVA-specific T cells. The second model involves intravenous injection of luciferase-expressing A20 to establish systemic lymphoma. Following the confirmation of systemic lymphoma development by live-animal bioluminescence imaging, animals will be randomly assigned to four different groups and systemically treated with PBS, αCD47, CpG or lead αCD47-CpG (at MTD) using the identical dose schedule employed in the flank tumor model study described above. Animals will then be monitored for body weight change and survival. Long-term survivors will be rechallenged with A20 cells to test whether the treatment mediates immunological memory.

### OTHER EMBODIMENTS

From the foregoing description, it will be apparent that variations and modifications may be made to the disclosure described herein to adopt it to various usages and conditions. Such embodiments are also within the scope of the following claims.

All citations to sequences, patents and publications in this specification are herein incorporated by reference to the same extent as if each independent patent and publication was specifically and individually indicated to be incorporated by reference.

1. A method of treating cancer comprising:
administering to a subject in need thereof, a composition comprising a therapeutically effective amount of chimeric molecule comprising a CD47 binding domain and an immunomodulatory agent wherein the immunomodulatory agent modulates innate and/or adaptive immune response(s), thereby treating cancer.

2. The method of claim 1, wherein the CD47 binding domain comprises antibodies, antibody fragments, single chain antibodies, humanized antibodies and antibody fragments; camelized antibodies and antibody fragments, human antibodies and antibody fragments, monospecific or bispecific antibodies, disulfide stabilized Fv fragments, scFv tandems, diabodies, tribodies or tetrabodies, peptoids, peptide or nucleic acid aptamers, antibody mimetics or combinations thereof.

3. The method of claim 2, wherein the antibody mimetics comprise DARPins, affibody molecules, affilins, affitins, anticalins, avimers, fynomers, Kunitz domain peptides or monobodies.

4. The method of claim 3, wherein the single chain antibodies or fragments comprise Fv fragments; single chain Fv (scFv) fragments, Fab′ fragments, F(ab′)₂ fragments, Fv, Fab′-SH, single-domain V_{H} single antibodies or combinations thereof.

5. The method of claim 1, wherein the immunomodulatory agent is a Toll-like receptor (TLR) agonist.

6. The method of claim 5, wherein the TLR comprises TLR2, TLR3, TLR4, TLR7, TLR8, TLR9 or combinations thereof.

7. The method of claim 6, wherein the TLR is TLR9.

8. The method of claim 7, wherein the TLR9 agonist comprises Poly-ICLC, LPS, MPL, CpG ODN or small molecules.

9. The method of claim 1, wherein the TLR agonist is conjugated to the CD47 binding molecule via a linker or a covalent bond.

10. The method of claim 8, wherein the covalent bond is a disulfide bond or any environmentally sensitive bond.

11. The method of claim 1, further comprising treatment with a secondary therapeutic agent.

12. The method of claim 11 wherein a secondary therapeutic agent comprises a chemotherapeutic agent, cytokines, an inhibitor of immune checkpoint suppressive activity, an agonist of immune stimulatory activity, a growth inhibitory agent, a targeted therapeutic agent, a T cell expressing a chimeric antigen receptor, an antibody or antigen-binding fragment thereof, an antibody-drug conjugate, an angiogenesis inhibitor, an antineoplastic agent, a cancer vaccine, an adjuvant, radiotherapy, or combinations thereof.

13. A composition comprising an anti-CD47 binding molecule conjugated to at least one Toll-like receptor (TLR) agonist.

14. The composition of claim 13, wherein the TLR comprises TLR2, TLR3, TLR4, TLR7, TLR8, TLR9 or combinations thereof.

15. The composition of claim 14, wherein the TLR is TLR9.

16. The composition of claim 15, wherein the TLR9 agonist comprises Poly-ICLC, LPS, MPL, CpG ODN or small molecules.

17. The composition of claim 15, wherein the TLR agonist is conjugated to the CD47 binding molecule via a linker or a covalent bond.

18. A composition comprising an anti-CD47 binding domain and one or more immunomodulatory agents.

19. The composition of claim 18, wherein the one or more immunomodulatory agents modulate an innate immune response, an adaptive immune response or the combination thereof.

20. The composition of claim 18, wherein the one or more immunomodulatory agents comprise a CD47 binding domain, a Toll-like receptor (TLR) agonist, Inducible T cell Co-stimulator (CD278), OX40 (CD134), 41 BB, Glucocorticoid-induced Tumor Necrosis Factor Receptor (GITR), CD40, CD27, stimulator of interferon (IFN) genes (STING) agonists or combinations thereof.
